(19) United States
(12) Patent Application Publication
(10) Pub. No.: US 2022/0167987 A1
(43) Pub. Date: Jun. 2, 2022

(54) APPARATUS, SYSTEM, AND METHOD FOR WOUND CLOSURE

(71) Applicant: KCI LICENSING, INC., San Antonio, TX (US)

(72) Inventor: Christopher Brian LOCKE, Bournemouth (GB)

(21) Appl. No.: 17/440,002

(22) PCT Filed: Mar. 26, 2020
(86) PCT No.: PCT/US2020/024975

§ 371 Date: Sep. 16, 2021

Related U.S. Application Data
(60) Provisional application No. 62/824,371, filed on Mar. 27, 2019.

(51) Int. Cl. A61B 17/08

(52) U.S. Cl. CPC A61B 17/083; A61B 2017/00951

(57) ABSTRACT

This disclosure describes devices, systems, and methods related to a wound closure device. An example of a wound closure device includes a plurality of base members configured to adhere to a tissue site and one or more attachment members for use in combination to create mechanical tension on the edges of a wound to facilitate closure of the wound. The plurality of base members include a first base member that includes light switchable adhesive. The light switchable adhesive is configured to transition from a first state to a second state, and the light switchable adhesive has a first peel strength in the first state that is greater than a second peel strength of the light switchable in the second state. The one or more attachment members are configured to couple to two or more base members of the plurality of base members.

### CROSS-REFERENCE WITH RELATED APPLICATIONS

This application claims priority to and the benefit of U.S. Provisional Application No. 62/824,371, filed Mar. 27, 2019, the contents of which is incorporated into the present application in its entirety.

### TECHNICAL FIELD

Aspects of the present disclosure relate generally to a wound closure device, and more specifically, but not by way of limitation, to a wound closure device with a light switchable adhesive.

### BACKGROUND

Wound closure devices, such as mechanical wound closure devices, are used to provide tension to a tissue site to assist in closing wounds and surgical openings. For example, two strips are placed on opposite sides of a wound or opening and are drawn together by a linking member to create tension that biases the skin together to keep the wound or opening closed. Conventional wound closure devices use hooks that are inserted into the skin and/or muscle or high tact and peel strength adhesives to secure the strips to the tissue site to generate the requisite tension forces used to close wounds and surgical openings. However, wound closure devices that use hooks, cause pain to the patient, require pain medication to reduce patient discomfort from the pain, can cause tearing in the skin and/or muscle, or any combination thereof. Additionally, wound closure devices that use high tact and peel strength adhesives (e.g., certain acrylic adhesives) are painful to remove and can cause damage to the skin (e.g., localized trauma) during removal. Examples of such damage include delamination of skin tissue and erythema. This is particularly true for a patient with a long term condition that requires an adhesive closure or dressing to be applied to the same part of the body repeatedly over a prolonged period, such as a stoma patient. It is also true for patients with fragile skin, especially the elderly and children, immunocomprismised patients, etc. Low tact and/or low peel strength adhesives (e.g., silicone based adhesives), which may not cause as much pain or damage to the patient as compared to high tact and peel strength adhesives, are not capable of generating sufficient tension and would peel off the tissue site before the requisite tension to close the wound or opening is created. Thus, conventional wound closure devices can be painful to apply, use, and remove and can cause skin trauma (e.g., puncture wounds, delamination, irritations, etc.).

### SUMMARY

This disclosure describes devices, systems, and methods related to wound closure and/or a wound closure device (e.g., a mechanical wound closure device). A mechanical wound closure device is a device that aids in the closing of a wound by manipulating the wound (e.g., edges thereof). The devices, systems, and methods can use or include a light switchable adhesive, a strain indicator, or both. A light switchable adhesive (often referred to as switched or light switched adhesive) is a pressure sensitive adhesive that is “switchable” from a tacky state (e.g., a first state) to a non-tacky or low-tack state (e.g., a second state) in which the light switchable adhesive has a reduced peel strength relative to the peel strength of the first state of the light switchable adhesive before switching. To illustrate, light, such as ultraviolet light, triggers (e.g., activates) cross linking in the light switchable adhesive which effectively decreases the bond (and peel strength) of the light switchable adhesive and enables a component of a wound closure device to decouple from a tissue site with reduced force. After the light switchable adhesive is switched from the first state to the second state by crosslinking, the light switchable adhesive becomes brittle and fragile, and the light switchable adhesive cannot be “uncrosslinked” or “unswitched.”

The light switchable adhesive may be coupled to one or more base members of the wound closure device, which are coupled to each other by attachment members. To illustrate, the light switchable adhesive may be employed on a base member of the wound closure device. An exemplary wound closure device may include one or more base members with light switchable adhesive. A light switchable adhesive can be employed on a particular base member in various ways, as described herein, to form a bond between the base member and a tissue site with a desired bond/peel strength. Additionally, the light switchable adhesives can be activated, i.e., switched or transitioned, in various ways, as described herein, to facilitate removal of the base member from the tissue site.

In some implementations, a light switchable adhesive may be activated by use of a dedicated light source. Additionally or alternatively, the light switchable adhesive may be activated by exposure of the light switchable adhesives to ambient light, such as by removal of a cover film or shroud. Thus, when the light switchable adhesive is in the first state, the wound closure device has a strong enough bond to the tissue site to generate the requisite tension for closing a wound or surgical opening, and when the light switchable adhesive is activated or switched to the second state, the peel strength of the light switchable adhesive is reduced which enables removal of the wound closure device, such as pain and trauma free removal of the wound closure device.

In some implementations, the wound closure device may include a ratchet mechanism to secure the attachment member or linking member to one or more of the base members. For example, the attachment member or linking member includes multiple teeth and may correspond to a linear rack configured to mate or interface with a pawl included in base member. The pawl mates or engages (often referred to as “clicks”) with the teeth of the linear rack as the attachment member is fed through the base member, and the pawl prevents the attachment member from being removed from the base member in opposite the fed direction. In other implementations, the wound closure device includes another type of locking or restraining mechanism to secure the attachment member or linking member to one or more of the base members, such as bar and clasp, a screw, tension cables, etc.

In some implementations, the wound closure device may include one or more indicators, such as one or more strain indicators, to indicate a tension level. For example, an attachment member or linking member that is coupled to multiple base members of the wound closure device may include a portion of flexible material that deforms under stress (e.g., tension) to provide a visual indication. To illustrate, deformation of the portion of flexible material may indicate an amount of tension or a tension level, such as a tension level greater than or equal to a threshold tension leave, for wound closure. For example, deformation of the portion of flexible material may provide the indication by changing color, shape, or both. In other implementations, the wound closure device includes a gauge, such as a strain gauge, to measure and output a strain value.

In some implementations, the wound closure device is used in conjunction with a dressing and/or therapy system. For example, the wound closure device may be applied to the tissue site, and then a wound dressing or negative pressure dressing may be applied over the wound closure device.

Thus, the wound closure device of the present disclosure is configured to mechanically close or aid in the closure of a wound or opening to promote healing. The wound closure device of the present disclosure can be positioned or arranged such that the wound closure device can treat different size wounds, multiple wounds, wounds with sutures, etc. By inclusion of a light switchable adhesive into base members of the wound closure device, the wound closure device can be more easily removed as compared to wound closure devices that include hooks inserted into the patient or that include high tact and peel strength adhesives. Additionally or alternatively, by inclusion of one or more strain indicators, the wound closure device can indicate a tension (e.g., range of tensions) or a tension states to a patient or care provider to enable the patient or care provider to set the wound closure device at a designed or desired amount of tension. Furthermore, the one or more strain indicators can also help a patient or care provider monitor an amount of tension generated by wound closure device during operation. In some implementations, the attachment members can be adjusted or the base members re-used to change or adjust tension. Thus, wound closure device can be adjusted to keep the designed or desired amount of tension throughout use of the wound closure device. Accordingly, the wound closure device may enable improved wound closure, thereby advancing patient comfort and confidence in the treatment.

Some embodiments of the present apparatuses (e.g., a wound closure device) comprise: a plurality of base members configured to adhere to a tissue site, the plurality of base members include a first base member including light switchable adhesive configured to transition from a first state to a second state, and the light switchable adhesive has a first peel strength in the first state that is greater than a second peel strength of the light switchable in the second state; and one or more attachment members configured to couple to two or more base members of the plurality of base members. In some implementations, the first base member further includes a pawl, and a particular attachment member of the one or more attachment members comprises a plurality of teeth configured to interlock with the pawl.

In some of the foregoing embodiments of the present apparatuses, the particular attachment member is coupled to a second base member of the plurality of base members. In some implementations, the particular attachment member is removably coupled to a second base member of the plurality of base members.

In some of the foregoing embodiments of the present apparatuses, the first base member further comprises: a polymer layer coupled to the light switchable adhesive and configured to pass light to the light switchable adhesive to transition the light switchable adhesive from the first state to the second state; and a protective film removably coupled to the polymer layer and configured to block ambient light from the light switchable adhesive. In some implementations, the polymer layer is optically transparent. In some implementations, the polymer layer is configured to diffuse the light to pass the light to the light switchable adhesive.

In some of the foregoing embodiments of the present apparatuses, the polymer layer is configured to pass (e.g., transmit) light having a wavelength between 10 nanometers and 500 nanometers. In some implementations, the protective film is configured to block or filter light having a wavelength between 10 nanometers and 500 nanometers. Additionally, or alternatively, the one or more attachment members comprise acrylonitrile butadiene styrene (ABS).

In some of the foregoing embodiments of the present apparatuses, the present apparatuses further comprise an indicator configured to provide a first visual indication when tension associated with a particular attachment member of the one or more attachment members is greater than or equal to a first threshold. In some implementations, the particular attachment member includes the indicator, wherein the indicator comprises an elastic polymer, and the indicator is configured to deform responsive to a force resulting from feeding the particular attachment member into at least one base member of the plurality of base members.

In some of the foregoing embodiments of the present apparatuses, the indicator comprises an elastic polymer, and the indicator is configured to deform responsive to a force resulting from feeding the particular attachment member into at least one base member of the plurality of base members. In some implementations: the indicator is configured to deform to transition between a first state and a second state, when the indicator is in the first state, the indicator is configured to indicate a first tension state, and when the indicator is in the second state, the indicator is configured to indicate a second tension state.

In some of the foregoing embodiments of the present apparatuses, the first state comprises an undeformed state, and the second state comprises a deformed state. In some implementations, the indicator has a first color or marking in the first state and a second color or marking different from the first color or marking in the second state.

In some of the foregoing embodiments of the present apparatuses, the indicator is configured to provide a second visual indication when the tension of the particular attachment member is greater than or equal to a second threshold. In some implementations: the indicator is configured to deform to transition between the second state and a third state, the indicator has a third color or marking that is different from the first color or marking and the second color or marking, and the first state corresponds to a first range of tensions, the second state corresponds to a second range of tensions, and the third state corresponds to a third range of tensions.

In some of the foregoing embodiments of the present apparatuses, the second visual indication indicates an over-tension state. In some implementations, the indicator is configured to transition to the second state between 4-8 Newtons. In some of the foregoing embodiments of the present apparatuses, the indicator includes a material that has a hardness in the range of 50 Shore A to 80 Shore A.

In some of the foregoing embodiments of the present apparatuses, the present apparatuses further comprise a second indicator configured to deform to indicate a third tension state. In some implementations, the indicator and the second indicator are integrated into the particular attachment member, and the indicator comprises a first portion of the particular attachment member and the second indicator comprises a second portion of the particular attachment member.

In some of the foregoing embodiments of the present apparatuses, a particular attachment member of the one or more attachment members comprises a strain gauge configured to output a strain value of the particular attachment member. In some of the foregoing embodiments of the present apparatuses, the present apparatuses further comprise a layer removably coupled to a particular side of the light switchable adhesive, the particular side of the light switchable adhesive configured to couple to the tissue site.

In some of the foregoing embodiments of the present apparatuses, the present apparatuses further comprise a shroud configured to be coupled to a patient and configured to block ambient light from the light switchable adhesive. In some of the foregoing embodiments of the present apparatuses, the first base member further includes adhesive. Additionally, or alternatively, the plurality of base members include a second base member that includes second light switchable adhesive.

Some embodiments of the present methods of attaching a wound closure device comprise: attaching a first base member to a first portion of a tissue site via a light switchable adhesive; attaching a second base member to a second portion of the tissue site; and coupling the first base member to the second base member via an attachment member. In some implementations, the attachment member comprises a strain indicator or a strain gauge.

In some of the foregoing embodiments of the present methods, the methods further comprise, prior to attaching the first base member, removing a cover film from the first base member. In some implementations, the methods further comprise, after coupling the first base member to the second base member, removing a protective film from first base member, wherein removing the protective film exposes the light switchable adhesive to ambient light.

In some of the foregoing embodiments of the present methods, the methods further comprise attaching a third base member to a third portion of the tissue site via a second light switchable adhesive. In some implementations, the methods further comprise a fourth base member and a second attachment member, wherein the third base member is oriented in parallel with the first base member and is coupled to the fourth base member via the second attachment member.

In some of the foregoing embodiments of the present methods, the third base member is aligned with the first base member lengthwise and is positioned such that third base member is coupled to the first base member via the attachment member. In some implementations, the third base member is aligned with the first base member and the second base member and is positioned such that third base member is coupled to the first base member and the second base member via the attachment member.

In some of the foregoing embodiments of the present methods, attaching the first base member includes attaching the light switchable adhesive to a tissue site and attaching the first base member to the light switchable adhesive attached to the tissue site.

In some of the foregoing embodiments of the present methods, the methods further comprise tightening the attachment member based on a strain indicator or a strain gauge of the attachment member. In some implementations, the methods further comprise ceasing the tightening of attachment member based on the strain indicator or the strain gauge of the attachment member. Additionally, or alternatively, the methods further comprise loosening the attachment member.

Some embodiments of the present methods comprise: receiving UV light from a UV device at a light switchable adhesive of a base member of a wound closure device; and responsive to receiving the UV light, transitioning from a first state to a second state by the light switchable adhesive. In some implementations, the methods further comprise debonding, by the light switchable adhesive, the base member from a tissue site, wherein the second state has a lower peel strength than the first state. Additionally, or alternatively, the methods further comprise, responsive to the UV light, changing color by the light switchable adhesive.

In some of the foregoing embodiments of the present methods, the methods further comprise, prior to receiving the UV light, bonding, by the light switchable adhesive, the base member to a tissue site. In some implementations, the methods further comprise, receiving the UV light at a second light switchable adhesive of a second base member of the wound closure device. Additionally, or alternatively, the methods further comprise, receiving the UV light to a third light switchable adhesive of a dressing associated with the wound closure device.

Some embodiments of the present methods of forming a wound closure device comprise: forming a light blocking layer; forming a non-light blocking layer; coupling the light blocking layer to the non-light blocking layer to form a base member; and applying light switchable adhesive to a portion of the non-light blocking layer. In some implementations, the methods further comprise coupling an attachment member to the base member.

In some of the foregoing embodiments of the present methods, coupling the attachment member to the base member includes bonding to the base member by an adhesive. In other implementations, coupling the attachment member to the base member includes bonding the attachment member to the base member by a molding process or forming the attachment member on the base member.

In some of the foregoing embodiments of the present methods, coupling the attachment member to the base member includes coupling the attachment member to the light blocking layer. In some implementations, coupling the attachment member to the base member includes coupling the attachment member between the light blocking layer and the non-light blocking layer.

In some of the foregoing embodiments of the present methods, the attachment member includes a strain indicator. In some implementations, the methods further comprise forming the attachment member by a twin-shot molding process. In other implementations, the methods further comprise coupling a strain gauge to the attachment member.

Some embodiments of the present kits (e.g., a kit for wound closure devices) comprise: a plurality of base members configured to adhere to a tissue site, the plurality of base members include a first base member including light switchable adhesive, the light switchable adhesive configured to transition from a first state to a second state. In some implementations, the kits further comprise a package that includes the plurality of base members and one or more attachment members configured to couple to two or more base members of the plurality of base member.

Some embodiments of the present kits (e.g., a kit for wound closure devices) comprise: one or more attachment members configured to couple to two or more base members, the one or more attachment members including an indicator configured to provide a first visual indication when tension associated with the particular attachment member is greater than or equal to a first threshold. In some implementations, the kits further comprise a package that includes the one or more attachment members and a plurality of base members configured to adhere to a tissue site, the plurality of base members include a first base member including light switchable adhesive, the light switchable adhesive configured to transition from a first state to a second state.

Some embodiments of the present systems (e.g., a therapy system) comprise: a wound closure device of any of the foregoing embodiments; and a light source configured to activate the light switchable adhesive of the wound closure device. In some implementations, the systems further comprise a wound dressing configured to at least partially cover the wound closure device.

In some of the foregoing embodiments of the present systems, the wound dressing comprises a viewing member to enable viewing of a wound, a strain indicator of the wound closure device, or both. In some implementations, the viewing member comprises a window. Additionally, or alternatively, the window comprises a transparent material or a translucent material such that a wound, the strain indicator, or both, are visible via the window.

In some of the foregoing embodiments of the present systems, the systems further comprise a therapy device configured to be coupled to the wound dressing via one or more tubes and configured to provide therapy to a wound.

As used herein, the term “switchable” will be used to refer to adhesives which can be changed from a high tack and/or peel strength state to a low tack and/or peel strength state (e.g., non-tacky state). Recognizing that the expression “low tack and/or peel strength” is a relative term, it will be defined here as meaning a condition of a minimum reduction in tackiness which the adhesive reaches after switching from the high tack and/or peel strength state. The reduction in tack or peel force may be as great as 99% or as little as 30%. Typically, the reduction in tack or peel force is between 70%. and 90%.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

A further understanding of the nature and advantages of the present disclosure may be realized by reference to the following drawings. The following drawings illustrate by way of example and not limitation. For the sake of readability and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a perspective view of an example of a system for wound closure;

FIG. 1B is a cross-sectional view of an example of a base member of FIG. 1A;

FIG. 2 is a perspective view of another example of wound closure device of FIG. 1A;

FIG. 3 is a perspective view of another example of wound closure device of FIG. 1A;

FIGS. 4A-4F are each a cross-sectional view of an example of a base member of FIG. 1A;

FIG. 5A is a top view of an example indication of the strain indicator of FIG. 1A;

FIG. 5B is a top view of an example second indication of the strain indicator of FIG. 5A;

FIG. 5C illustrates example indications of the strain indicator of FIG. 1A;

FIG. 6 is diagram of an example of a system for wound therapy including a wound closure device;

FIG. 7 is a diagram of a kit for wound closure devices;

FIG. 8 is a flowchart illustrating an example of a method of attaching of a wound closure device;

FIG. 9 is a flowchart illustrating an example of a method of operating a wound closure device; and

FIG. 10 is a flowchart illustrating an example of a method of forming a wound closure device.

### DETAILED DESCRIPTION

As used herein, the terms “tissue site” and “target tissue” as used herein can broadly refer to a wound (e.g., open or closed), a tissue disorder, and/or the like located on or within tissue, such as, for example, bone tissue, adipose tissue, muscle tissue, neural tissue, dermal tissue, vascular tissue, connective tissue, cartilage, tendons, ligaments, and/or the like. The terms “tissue site” and “target tissue” as used herein can also refer to a surrounding tissue area(s) and/or areas of tissue that are not necessarily wounded or exhibit a disorder, but include tissue that would benefit from tissue generation and/or tissue that may be harvested and transplanted to another tissue location. The terms “tissue site” and “target tissue” may also include incisions, such as a surgical incision. In some implementations, “target tissue” may correspond or refer to a wound, and “tissue site” may correspond or refer to a tissue area(s) surrounding and including the target tissue. Additionally, the term “wound” as used herein can refer to a chronic, subacute, acute, traumatic, and/or dehisced incision, laceration, puncture, avulsion, and/or the like, a partial-thickness and/or full thickness burn, an ulcer (e.g., diabetic, pressure, venous, and/or the like), flap, and/or graft. A wound may include chronic, acute, traumatic, subacute, and dehisced wounds, surgical openings, partial-thickness burns, ulcers (such as diabetic, pressure, or venous insufficiency ulcers), flaps, grafts, and fistulas, for example.

The term “positive-pressure” (or “hyperbaric”) as used herein generally refers to a pressure greater than a local ambient pressure, such as the ambient pressure in a local environment external to a sealed therapeutic environment (e.g., an internal volume). In most cases, this positive-pressure will be greater than the atmospheric pressure at which the patient is located. Alternatively, the positive-pressure may be greater than a hydrostatic pressure associated with tissue at the tissue site. Unless otherwise indicated, values of pressure stated herein are gauge pressures. References to increases in positive-pressure typically refer to an increase in absolute pressure, and decreases in positive-pressure typically refer to a decrease in absolute pressure. Additionally, the process of increasing pressure may be described illustratively herein as “applying”, “delivering,” “distributing,” “generating”, or “providing” positive-pressure, for example.

The term “reduced-pressure” (and “negative-pressure” or “hypobaric”) as used herein generally refers to a pressure less than a local ambient pressure, such as the ambient pressure in a local environment external to a sealed therapeutic environment (e.g., an internal volume). In most cases, this reduced-pressure will be less than the atmospheric pressure at which the patient is located. Alternatively, the reduced-pressure may be less than a hydrostatic pressure associated with tissue at the tissue site. Unless otherwise indicated, values of pressure stated herein are gauge pressures. References to increases in reduced-pressure typically refer to a decrease in absolute pressure, and decreases in reduced-pressure typically refer to an increase in absolute pressure. Additionally, the process of reducing pressure may be described illustratively herein as “applying”, “delivering,” “distributing,” “generating”, or “providing” reduced-pressure, for example.

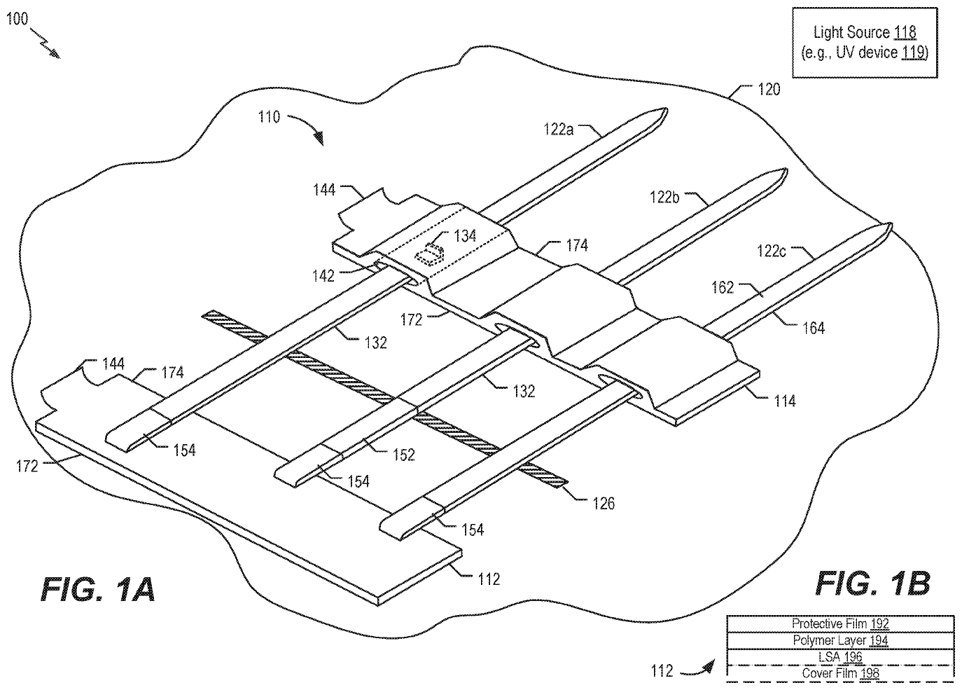

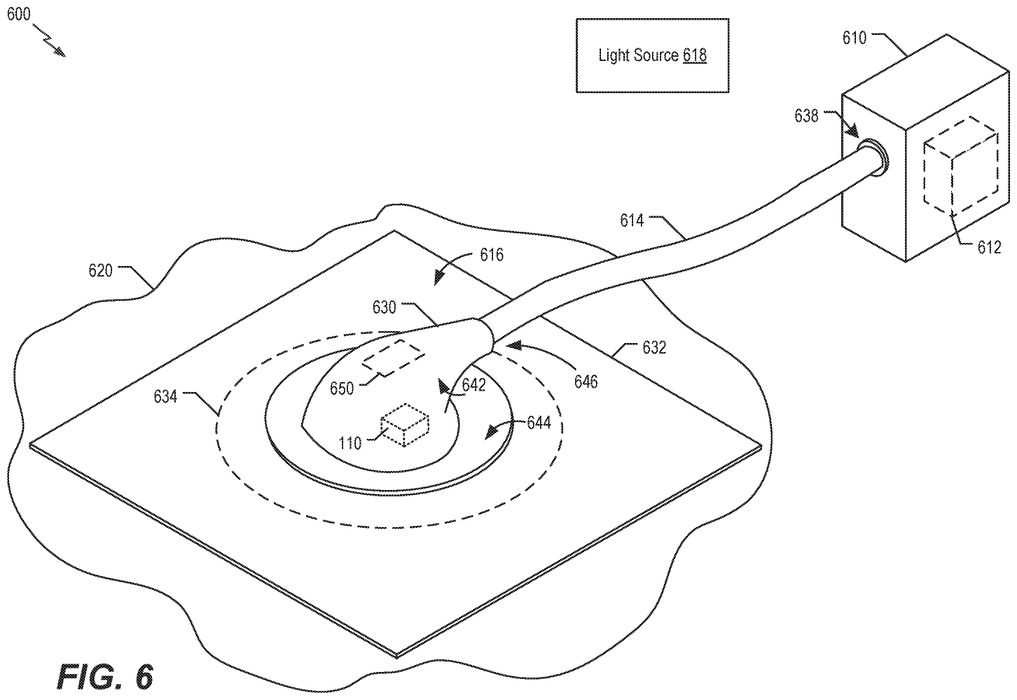

FIG. 1A shows a perspective view of an illustrative system 100 for wound closure, such as mechanical wound closure. System 100 includes a wound closure device 110 and may include a light source 118. System 100 is configured to provide tension at a tissue site 120 associated with a wound or an opening 126 in tissue of a patient. For example, wound closure device 110 is attached to target tissue near the wound or the opening 126 and is configured to generate and hold tension such that the wound or the opening 126 remains closed or “pinched” together. In some implementations, a dressing (e.g., a wound dressing) may be in fluid communication with tissue site 120 and may be in fluid communication with a therapy device via one or more tubes, as described further with reference to FIG. 6. In such implementations, wound closure device 110 functions to keep the wound or opening 126 closed and the therapy device and dressing provide therapy to the wound or opening 126. In some implementations, system 100 may include one or more components commercially available through and/or from KCI USA, Inc. of San Antonio, Tex., U.S.A., and/or its subsidiary and related companies (collectively, “KCI”).

In FIG. 1A, wound closure device 110 includes a plurality of base members 112, 114 and one or more attachment members 122a-c. The plurality of base members 112, 114 are coupled to target tissue near the wound or the opening 126. As illustrated in FIG. 1A, the wound or the opening 126 is linear or straight and the plurality of base members 112, 114 are positioned next to and parallel with the wound or the opening 126. In other implementations, the plurality of base member 112, 114 can be arranged in different ways, such as when a wound or the opening 126 has a different or non-linear shape. For example, one or more base members may be arranged radially, perpendicularly, diagonally, etc., or a combination thereof.

One or more of the plurality of base members 112, 114 are coupled to each other via one or more attachment members 122a-c. As illustrated in FIG. 1A, the one or more attachment members 122a-c each couple together first and second base members 112, 114. The plurality of base members 112, 114 include or more layers of materials, as illustrated in FIG. 1B.

Referring to FIG. 1B, one or more base members of the plurality of base members 112, 114 include a removable protective film, referred to as protective film 192, a polymer layer 194, and a light switchable adhesive (LSA) 196. As illustrated in FIG. 1B, first base member 112 includes protective film 192, polymer layer 194, LSA 196, and optionally cover film 198.

Protective film 192 is configured to be removed from polymer layer 194 while polymer layer 194 is bonded to tissue site 120. Protective film 192 is configured to block or filter light of a particular wavelength associated with activating the LSA 196, and polymer layer 194 is configured to pass or transmit the light of the particular wavelength associated with activating the LSA 196. For example, the protective film 192 is configured to block or filter UV light to blue light wavelengths and/or the polymer layer 194 is configured to pass UV light to blue light wavelengths. To illustrate, the protective film 192 is configured to block or filter light having a wavelength between 10 nanometers and 500 nanometers and/or the polymer layer 194 is configured to pass light having a wavelength between 10 nanometers and 500 nanometers. In other implementations, the light which is blocked or filtered by the protective film 192 and/or passed by polymer layer 194 includes or corresponds to visible light, a portion of the visible light spectrum, UV light, a portion of the UV light spectrum, or a combination thereof.

In a particular implementation, protective film 192 is configured to be permeable to air, to enable tissue of tissue site 120 to which the base member is bonded to “breathe.” Protective film 192 has a peel strength that is less than a peel strength of the LSA 196 in the first state. In a particular implementation, protective film 192 includes a tab 144 to enable easy removal of the protective film 192 from a particular base member. Tab 144 may extend outwards and/or upwards from the base member to facilitate removal or protective film 192 from polymer layer 194.

Protective film 192 and polymer layer 194 of base members may include an impermeable or semi-permeable, elastomeric material, as an illustrative, non-limiting example. In some implementations, protective film 192 and/or polymer layer 194 are liquid/gas (e.g., moisture/vapor) impermeable or semi-permeable. Additionally or alternatively, protective film 192 and/or polymer layer 194 include or are elastomeric material. “Elastomeric” means having the properties of an elastomer. For example, elastomer generally refers to a polymeric material that may have rubber-like properties. More specifically, an elastomer may typically have ultimate elongations greater than or equal to 100% and a significant amount of resilience. The resilience of a material refers to the material's ability to recover from an elastic deformation. Elastomers that are relatively less resilient may also be used as these elastomers. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In a particular implementation, protective film 192 includes polyurethane (PU), such as semi-rigid PU, and polymer layer 194 includes silicone or thermoplastic elastomers, such as Santoprene, a registered trademark of Exxon Mobil Corporation. In other implementations, protective film 192 includes or corresponds to cellulose.

In some implementations, polymer layer 194 is configured to diffuse light received from a top (e.g., when protective film 192 is removed) and/or a side to LSA 196. To illustrate, light received on a side polymer layer 194 is scattered as it passes through polymer layer 194 to distribute the light to a larger area on the opposite side. Additionally, or alternatively, polymer layer 194 is be formed of a thin, clear, flexible, breathable material with a high refractive index. One exemplary material for the cover film is polyurethane (PU).

LSA 196 is included or disposed on polymer layer 194 of at least one base member (e.g., 112) and is configured to generate a bond between the at least one base member and tissue site 120. LSA 196 may include or correspond to a light switchable adhesive as described in International Patent Application Nos. PCT/US2018/049388 and PCT/US 2018/060718, which are incorporated by reference herein to the extent they describes light switchable adhesives. LSA 196 includes one or more photo initiators and is configured to switch states upon exposure to light of a particular spectrum or wavelength. The photo initiators are configured to absorb light (of particular spectrum or wavelength) and cross link with each other and/or free radicals to reduce tackiness, increase brittleness, increase fragility, reduce ductileness, change color, or a combination thereof. Thus, LSA 196 transitions from a first state (e.g., high tack state) to a second state (e.g., a low tack, no tack, or cross linked state) upon exposure to light. Transitioning from the first state to the second state enables easy, pain and trauma free removal of the dressing and/or easy disconnection of a connection point. As an illustrative example, LSA 196 may have a peel strength of greater than or equal to 18 N in the first state and a peel strength of greater than or equal to 0.3 N in the second state. LSA 196 may include or correspond to a polyurethane (PU) or acrylic based light switchable adhesive.

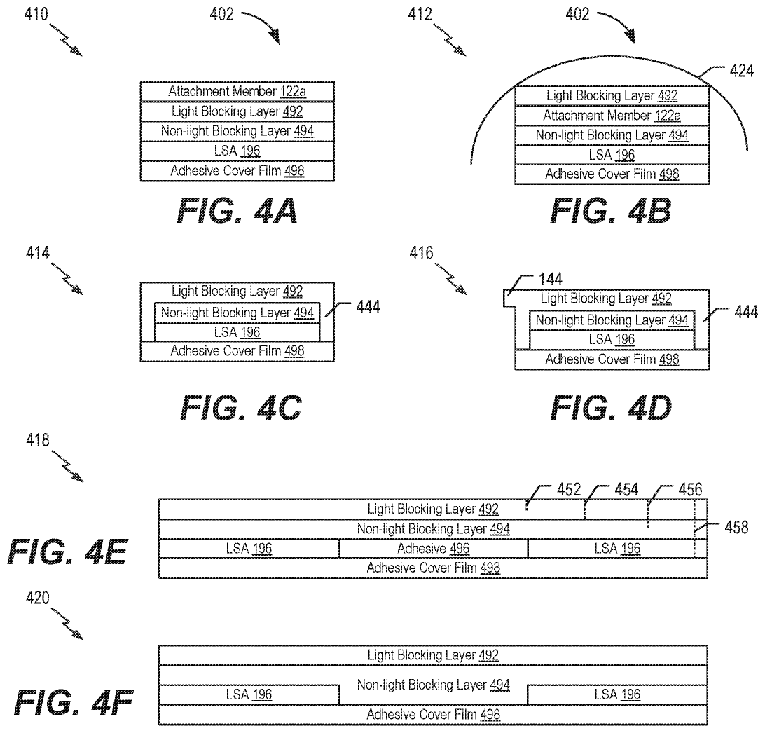

In some implementations, LSA 196 includes UV photo initiators and is configured to absorb UV light (light from at least a portion of the UV spectrum) and switch states. In other implementations, LSA 196 includes visible light photo initiators and is configured to absorb visible light (light from at least a portion of the visible light spectrum) and switch states. LSA 196 may be formed with one or more materials of the base members, such as co-extruded with one or more of 192, 194, 196. Alternatively, LSA 196 may be applied to polymer layer 194, e.g., extruded after formation of the polymer layer 194. In some implementations, LSA 196 is a coating or a pattern of coatings, as described further with reference to FIGS. 4E and 4F.

In some implementations, LSA 196 includes a UV marking additive. In a particular implementation, the UV marking additive includes or corresponds to an ultraviolet absorber (UV absorber). A UV absorber is a molecule used in organic or synthetic materials to absorb UV radiation. The UV absorbers are configured to absorb at least a portion of UV radiation of the UV spectrum and produce a visual indication, such as a color change. For example, UVA absorbers are configured to absorb UVA radiation, i.e., electromagnetic radiation having wavelengths between 300 and 400 nm. Additionally, or alternatively, one or more other layers of a base member may include a UV marking additive or another additive, such as a visible light additive. For example, protective film 192 and/or polymer layer 194 may include a marking additive. Such marking additives may produce a color change, produce text, produce a symbol, etc. to indicate light which may activate LSA 196 has been received.

In some implementations, LSA 196 has or is configured to provide a bond strength (e.g., peel strength) at least at or greater than, or substantially equal to any one of, or between two of: 10, 12, 14, 16, 18, or 20 N, in the first state. The bond may be formed by LSA 196 between polymer layer 194 and tissue, such as target tissue of tissue site 120. To illustrate, LSA 196 may have a bond strength as described above or may be applied such that a base member of wound closure device 110 has a bond strength as described above. In some implementations, the bond strength of the LSA 196 increases after application. For example, the bond strength of the LSA 196 may reach a maximum bond strength between 30 minutes to 2 hours after application. Additionally, or alternatively, LSA 196 has or is configured to provide a bond strength (e.g., peel strength) at least at or greater than, or substantially equal to any one of, or between two of: 0.3, 0.5, 1, 2, 3, 4, 5, 6, 8, or 10 N, in the second state after being exposed to light.

In some implementations, one or more base members of the plurality of base members 112, 114 further include a cover film 198. Cover film 198 (e.g., an adhesive cover film or layer) is positioned over or coupled to LSA 196 to protect LSA 196 from activation, i.e., receiving light and transitioning to the second state, and from dust or contamination. Cover film 198 is configured to be removed prior to application of base member to tissue site 120 and as such has a lower peel strength or bond strength to the LSA 196 than a peel strength or bond strength between the LSA 196 and the polymer layer 194. Cover film 198 may be formed of a thin, clear, flexible, breathable material with a high refractive index. One exemplary material for cover film 198 is polyurethane (PU).

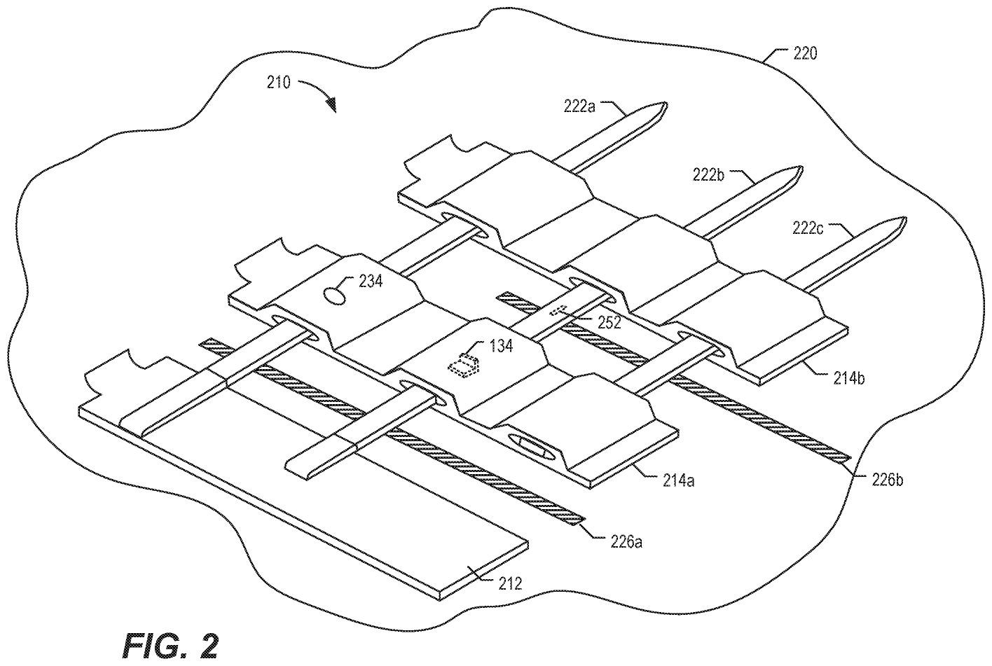

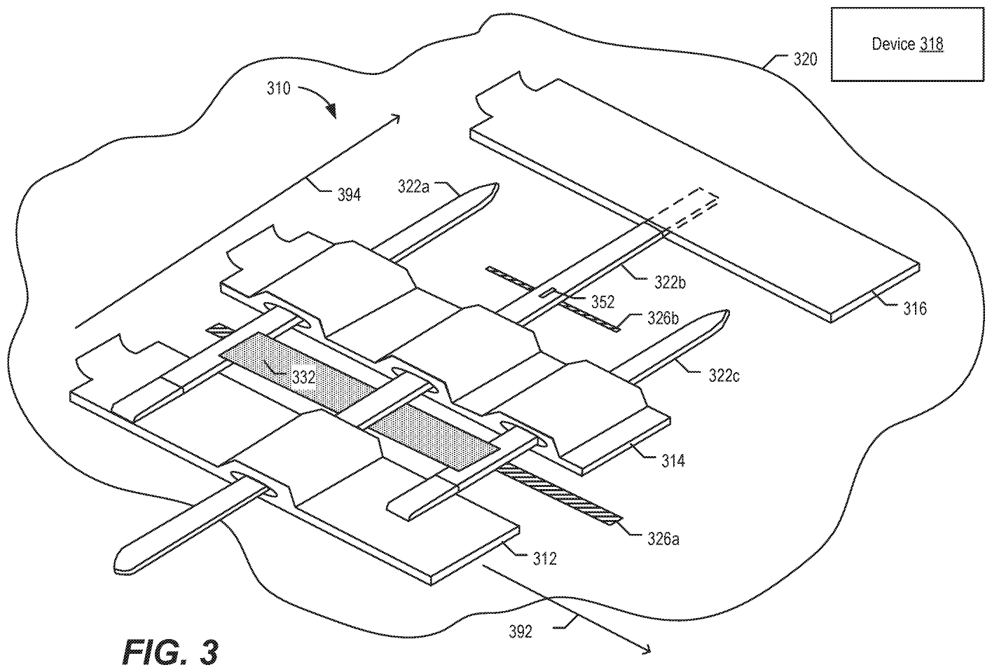

Referring to FIG. 1A, wound closure device 110 includes a first base member 112 of a first type and a second base member 114 of a second type. In FIG. 1A, first base member 112 corresponds to an anchor base member and each of the one or more attachment members 122a-c are coupled (e.g., attached or anchored) to the first base member 112. In some implementations, the one or more attachment members 122a-c may be bonded to the first base member 112. The one or more attachment members 122a-c may be coupled or bonded to the first base member 112 in different positions or in similar positions. For example, as illustrated in FIG. 1, attachment member 122a is attached near a proximal side 172 of first base member 112, while attachment members 122b-c are attached near a distal side 174 of first base member 112. Additional attachment or anchor locations are illustrated in FIGS. 2 and 3.

In FIG. 1A, second base member 114 corresponds to a ratchet base member and includes a component of a ratchet mechanism. In the example illustrated in FIG. 1A, second base member 114 defines one or more openings 142 configured to receive one or more attachment members 122a-c and include one or more pawls 134 configured to interlock with teeth 132 (e.g., ridges) of the one or more attachment members 122a-c, as described further with reference to FIG. 2. As illustrated in FIG. 1A, opening 142 is a channel (e.g., a through channel) having a first aperture on a proximal side 172 and a second aperture on a distal side 174. For example, the first aperture is located on an outer side surface (e.g., wound facing side surface) of second base member 114 and the second aperture is located on an outer side surface of second base member 114. The attachment members 122a-c are inserted into the first aperture on the proximal side 172, extend through the second base member 114, and protrude out from the second aperture on the distal side 174. Attachment members 122a-c include a top side 162 and a bottom side 164. Top side 162 faces away from wound 126 and bottom side 164 faces towards wound 126.

Attachment members 122a-c include or correspond to flexible, structural plastics, such as acrylonitrile butadiene styrene (ABS), polyester, etc. In some implementations, attachment members 122a-c are configured to provide and hold forces greater than or equal 20 N. In some implementations, attachment members 122a-c include an optically transparent or non-light blocking material. To illustrate, attachment members 122a-c enable light that would otherwise activate LSA 196 to pass thought the attachment members 122a-c. In other implementations, attachment members 122a-c include an opaque or light blocking material. To illustrate, attachment members 122a-c block or filter light that would otherwise activate LSA 196.

In some implementations, wound closure device 110 further includes one or more strain indicators 152, strain gauges, or a combination thereof. Strain indicator 152 includes an deformable/expandable member or structure, such as a flexible polymer member. In some implementations, strain indicator 152 may include a relatively soft elastomeric material, as compared to materials of the base members 112, 114 and other materials of the attachment members 122a-c. Examples of elastomers for strain indicator 152 may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In a particular implementation, silicone, a thermoplastic elastomer (TPE), such as Santoprene.

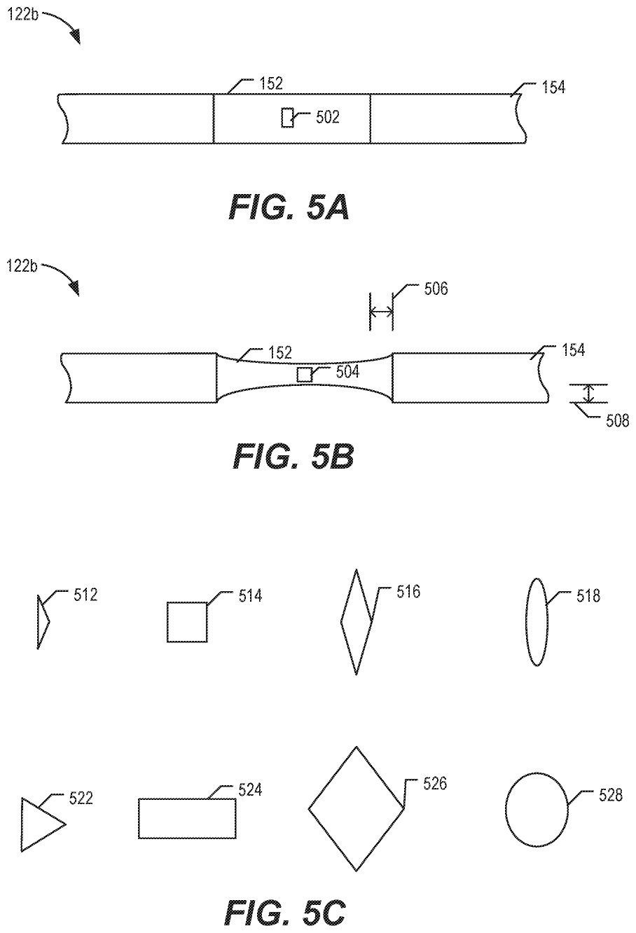

Strain indicator(s) 152 may be coupled to or incorporated into one or more attachment members 122a-c. As illustrated in FIG. 1A, strain indicator 152 is coupled to (or near) an anchor portion 154 of second attachment member 122b and proximate to wound or opening 126. When second attachment member 122b is fed through second base member 114 tension is created, which causes the strain indicator 152 to deform responsive to the tension. To illustrate, responsive to the tension created by the second attachment member 122b, strain indicator 152 deforms to elongate and produce a visual indication, as described further with reference to FIGS. 5A and 5B. In some implementations, strain indicator 152 is colored or has one or more markings. For example, the strain indicator 152 includes one or more colors or markings to indicate one or more positive-pressure states, as described further with reference to FIGS. 5A-5C.

Strain indicator 152 includes a flexible or compliant material, as described above, which is configured to deform under tension, such as when tension of the second attachment member 122b exceeds a threshold. For example, strain indicator 152 may be configured to deform to provide the visual indication between 2 and 20 Newtons. To illustrate, strain indicator 152 may be configured to provide the visual indication at a tension of greater than, or substantially equal to any one of, or between any two of: 2, 4, 6, 8, 10, 12, 14, 16, 18, or 20 N. The visual indication may indicate tension has been created at a level which promotes healing. Deforming to an extent such that a visual indication is provided may be referred to as deforming to a state or transitioning between states. Strain indicator 152 is configured to deform at a force or strain that is less than a peel strength of the LSA 196 and the bond strength of the base member, such that strain indicator 152 will deform before the base member is detached from tissue site 120.

As another example, strain indicator 152 may be configured to deform to provide the visual indication at tensions above 4 Newtons. To illustrate, strain indicator 152 begins deforming at less than 4 Newtons, and at 4 Newtons, strain indicator 152 deforms (or has deformed) to such a degree or extent that strain indicator 152 provides the visual indication. In other implementations, strain indicator 152 deforms to such a degree or extent that strain indicator 152 provides the visual indication at 8 N or above, 10 N or above, 12 N or above, etc. Thus, the visual indication may be associated with any pressure that is greater than a threshold pressure. In some implementations, the visual indication is associated with a specific range of pressures, and a second visual indication is associated with a second range of pressures. As illustrative, non-limiting examples, the range of pressures may include one of 2-10, 6-14, 10-20, 12-18, 12-20, 14-18, 16-20, 14-20 N, etc., and the second range of pressures may include one of 12-20, 18-14, 20-30, greater than 22, greater than 25 N, etc. In some implementations, the second range of pressures may include at least at or greater than, or substantially equal to any one of, or between two of: 12, 14, 16, 18, 19, 20, 21, 22, 23, 24, 25, 26, 27, 28, or 30 N.

In some implementations, strain indicator 152 has a hardness of 40 Shore A to 100 Shore A or includes a material that has a hardness of 40 Shore A to 100 Shore A. In a particular implementation, strain indicator 152 has a hardness of 50 Shore A to 80 Shore A or includes a material that has a hardness of 50 Shore A to 80 Shore A. As illustrative, non-limiting examples, strain indicator 152 comprises a silicone elastomer material, a fluorosilicone material, an ethylene propylene diene terpolymer (EPDM) material, a nitrile butadiene rubber (NBR) material, a thermoplastic elastomer material (e.g., Hytrel a registered trademark of E. I. Du Pont De Nemours and Company), a polyether block amide material (e.g., PEBAX a registered trademark of Arkema, Inc.), or a polyurethane (PU) material. In implementations where an attachment member includes a strain indicator 152, the attachment member can be made by twin shot molding (e.g., 2 shot molding) to form the attachment member from two different materials.

Light source 118 is configured to provide light to activate LSA 196 (photo initiators thereof) and cause LSA 196 to switch states. Light source 118 may include or correspond to the Sun, ambient lighting, a dedicated light device, such as an ultraviolet (UV) device 119 of FIG. 1A, or a combination thereof.

As illustrated in FIG. 1A, light source 118 includes UV device 119 configured to generate UV light to activate LSA 196 (photo initiators thereof) and cause LSA 196 to switch states. For example, UV device 119 includes or corresponds to a UV light source configured to generate light or electromagnetic radiation having a wavelength of 10-500 nanometers, such as UV light to blue light. In some implementations, UV device 119 may include or correspond to a UV torch. For example, UV torch may include one or more LEDs configured to generate incoherent light in the UV spectrum. In a particular implementation, UV torch generates light in a particular subspectrum of the UV spectrum, such as UVA or UVC.

In other implementations, UV device 119 may include or correspond to a UV Laser, such as a gas laser, a laser diode, a solid-state laser, an excimer laser, or a combination thereof. In some implementations, UV laser is configured to generate coherent light (e.g., a laser beam) having electromagnetic radiation of UV wavelengths. For example, UV laser is a UVA laser (315-400 nm), a UVB laser (280-315 nm), a UVC laser (100-280 nm), or an extreme UV laser (10-121 nm).

In some implementations, UV device 119 may be integrated with a component of system 100, such a base member. In such implementations, UV device 119 generates UV light and directs the UV light to a UV light passage in a base member. UV light passage (e.g., a channel, a window, or light passing or diffusing medium) is configured to direct the UV light to the LSA 196. UV light passage may optionally include one or more components to direct or focus the UV light, such as one or more lens, refractors, collimators, etc.

In other implementations, one or more base members include a port or a window (e.g., 650 of FIG. 6) configured to receive light from a light source or UV device 119. The port or window may include a cover or a film to reduce or prevent light from entering the port or window when the UV device 119 is not coupled to the port or the window is not in use. Alternatively, the port or window may be incorporated into polymer layer 194 and a portion of protective film 192 corresponds to the cover or film which can be moved or removed to access the port or window.

In other implementations, wound closure device 110 receives the UV light directly from a UV light source or from ambient light. In some such implementations, wound closure device 110 includes a shroud (e.g., 424, 616). The shroud is positioned such that the shroud blocks the LSA 196 from receiving light, such as ambient light. The shroud is formed of or includes a material that is configured to block (e.g., reflect or absorb) ambient light that would otherwise activate LSA 196. Thus, shroud enables the use of ambient light and/or does not require a dedicated light device, transporting light via components of a therapy system, or both. Accordingly, LSA 196 can be activated without a dedicated light device.

During operation of system 100, wound closure device 110 is coupled to tissue site 120. To illustrate, cover film 198 of first base member 112 is removed and first base member 112 is attached to first target tissue of tissue site 120 near or proximate to wound 126. As illustrated in FIG. 1A, first base member 112 is aligned lengthwise and in parallel with wound 126 on a first side of wound 126. Cover film 198 of second base member 114 is removed and second base member 114 is attached to second target tissue of tissue site 120 near or proximate to wound 126. As illustrated in FIG. 1A, second base member 114 is aligned lengthwise and in parallel with first base member 112 and wound 126 on a second side of wound 126 opposite the first side.

Attachment members 122a-c are used to couple together the first and second base members 112, 114. As illustrated in FIG. 1A, attachment members 122a-c are attached or bonded to first base member 112. Although anchor portion 154 of attachment member 122a is illustrated as partially extending over or attaching to first base member 112, in other implementations, anchor portion 154 extends over an entirety of first base member 112 or over an entirety of an overlap between attachment member 122a and first base member 112, similar to attachment member 222a and attachment member 222b of FIG. 2 respectively. Additionally, or alternatively, protective film 192 may extend over one or more attachment members 122a (e.g., anchor portions 154 thereof), as described further with reference to FIGS. 4A and 4B.

Attachment member 122a-c are inserted into corresponding openings 142 in second base member 114. Attachment members 122a-c are fed through the openings 142 until a designed or desired amount of tension is created between first and second base member and applied to wound 126. The tension applied to wound 126 may not be even or equal by design. First attachment member 122a may have a shorter length from first base member 112 to second base member 114 to generate more tension, as compared to third attachment member 122c. Thus, a patient or care provider can apply more tension to a particular portion of wound 126 to promote healing, reduce patient discomfort, or enable a wider range of motion by the patient.

In other implementations, attachment members 122a-c are not bonded to first base member 112 and attachment members 122a-c are inserted into cavities, openings (e.g., 142), hooks, eyes, or another restraining means of first base member 112 prior to insertion into second base member 114 or attachment members 122a-c are coupled to first base member 112 prior to insertion into second base member 114, such as by adhesive. For example, attachment members are inserted into openings (e.g., 142) of multiple base members 112, 114, as described further with reference to FIGS. 2 and 3.

In a particular implementation, one or more attachment members 122a-c include a strain indicator 152, and patient or care provider uses strain indicator 152 in determining an amount of tension. For example, after attachment members 122a-c are partially inserted, patient or care provider may feed attachment members 122a-c singularly or together while watching or monitoring deformation of strain indicator 152. When strain indicator 152 provides a particular visual indication, as described further with reference to FIGS. 5A-5C, patient or care provides stops feeding (e.g., tightening) one or more attachment members 122a-c. Additionally, during operation if strain indicator 152 produces another visual indication, as described further with reference to FIGS. 5A-5C, patient or care provider may adjust attachment member(s) 122a-c. For example, one or more of attachment members 122a-c may be tightened by feeding more of one or more of attachment members 122a-c into and through second base member 114, one or more of attachment members 122a-c may be loosened by removing or releasing one or more of attachment members 122a-c from one or more base members 112, 114, such as from second base member 114 as further described with reference to FIG. 2.

When strain indicator 152 deforms to such a degree to provide the visual indication, the strain indicator 152 is in a deformed state (e.g., a second state). The visual indication and/or second state may indicate tension is being applied to tissue site 120 and/or operational tension has been reached. In some implementations, when tension exceeds a threshold tension value, the strain indicator 152 deforms further (relative to an amount of deformation associated with visual indication) to provide a second visual indication. For example, the strain indicator 152 deforms to transition to an over-deformed state or a fully-deformed state (e.g., a third state), and strain indicator 152 indicates an over-tensioned state. Alternatively, a second strain indicator (e.g., similar to strain indicator 152) may be incorporated into the same attachment member as strain indicator 152 or another attachment member and is configured to indicate an over-tensioned state. Avoiding over-tensioned state states may increase wound recovery by avoiding an over-tensioned state in which capillaries of tissue site 120 may begin to close or delamination of tissue occurs in tissue site 120.

Thus, wound closure device 110 is configured to mechanically close a wound or opening in tissue site 120 to promote healing and, by inclusion of LSA 196, can be more easily removed as compared to wound closure devices that include high tact and peel strength adhesives. Additionally, by inclusion of strain indicator(s), wound closure device 110 can indicate tension states to a patient or care provider to enable wound closure device 110 to generate a designed or desired amount of tension. Furthermore, strain indicator(s) can also help a patient or care provider monitor an amount of tension generated by wound closure device 110 during operation and enable wound closure device 110 to be adjusted to keep the designed or desired amount of tension throughout use of the wound closure device. Accordingly, wound closure device 110 enables mechanical wound closure with adhesive that include a low tact and/or peel strength state and without using and/or independent of hooks or devices which are inserted into tissue site 120 thereby avoiding or limiting tissue damage at tissue site 120, pain medication, and patient discomfort. Accordingly, wound closure device 110 and/or light source 118 may enable improved wound closure through use of system 100, thereby advancing patient comfort and confidence in the treatment.

FIGS. 2 and 3 illustrate additional examples of wound closure devices. Wound closure devices 210 and 310 of FIGS. 2 and 3 may include or correspond to wound closure device 110 of FIG. 1A. Referring to FIG. 2, a perspective view of an example of a wound closure device 210 is shown. Wound closure device 210 includes base members 212, 214a, and 214b and attachment members 222a-c. Base members 212, 214a, 214b may include or correspond to base members 112, 114. Attachment members 222a-c may include or correspond to attachment members 122a-c.

In some implementations, one or more base members of base members 212, 214a, and 214b include a release button 234, tab, lock, or another release mechanism. The release button 234 is configured to operate a release mechanism to enable backwards movement or removal of an attachment member. Backwards movement or removal of attachment member can be used to reduce or relieve tension. As illustrated in FIG. 2, the release button 234 depresses or deforms a release mechanism (e.g., pawl 134) such that the release mechanism disengages from the attachment member, such as teeth (e.g., 132) thereof. Thus, a ratchet mechanism that normally only allows one-way travel or feeding, can travel in two directions and be adjusted. In other implementations, an adjustable attachment member can be used. For example, a threaded adjustment member (e.g., screw) or a rod can be used as for one or more of attachment members 222a-c. To illustrate, when a rod is used as an attachment member 222a-c, tension clips coupled to or integrated with one or more base members 212, 214a, 214b can be used to control positioning of the rod and tension generated thereby.

As illustrated in FIG. 2, tissue site 220 includes a first wound 226a and a second wound 226b. First wound 226a and second wound 226b may include or correspond to surgical openings. The surgical openings may be closed with stiches or a tissue safe topical adhesive, such as Dermabond, a registered trademark of Ethicon. Wounds 226a and/or 226b may include or correspond to wound 126. Tissue site 220 may include or correspond to tissue site 120.

First base member 212 is positioned proximate to first wound 226a, second base member 214a is positioned between wounds 226a and 226b, and third base member 214b is positioned proximate to second wound 226b. Attachment members 222a-c couple each of the base members 212, 214a, and 214b to each other. To illustrate, attachment members 222a-b are attached to first base member 212 and extend through corresponding openings defined by second and third base members 214a and 214b. Attachment member 222c is coupled to second and third base members 214a and 214b.

During operation, base members 212, 214a, and 214b, are adhered to tissue site 220 by LSA, such as LSA 196, as described with reference to FIG. 1A. Attachment members 222a-b, which are coupled to first base member 212, are fed through openings defined by second and third base members 214a, 214b. Attachment member 222c is fed through openings (e.g., 142) defined by second and third base members 214a, 214b. For example, attachment member 222c is fed across the wound 226b from either of second base members 214a or the third base member 214b. Alternatively, attachment member 222c is fed from the wound 226b to both the second base member 214a and the third base member 214b (e.g., away from or inside out relative to the wound 226b). In some such implementations, the attachment member 222c includes two sets of teeth (e.g., 132). To illustrate, one set of teeth oriented in a first direction and configured to interlock with a pawl of the second base member 214a and another set of teeth oriented in a second direction opposite the first direction and configured to interlock with a pawl of the third base member 214b. Thus, anchors (e.g., 154) of attachment members 222c can be removed or eliminated.

As described with reference to FIG. 1A, one or more strain indicators 252 (e.g., 152) or strain gauges (e.g., 352) can be used to indicate when an desired amount of force or tension is created. Insertion of attachment members 222a-b into second base member 214a, generates tension between second base member 214a and first base member 212, and insertion of attachment members 222a-b into third base member 214b generates tension between third base member 214b and second base member 214a and between third base member 214b and first base member 212. Insertion of attachment member 222c into second base member 214a and third base member 214b, generates tension between second base member 214a and third base member 214b.

In some implementations, a base member does not include a ratchet or securing mechanism for each attachment member. For example, as illustrated in FIG. 2, third base member 214b may not have a ratchet mechanism (e.g., pawl 134) for first attachment member 222a. Thus, no tension is created in tissue site 220 between second and third base members 214a and 214b by first attachment member 222a. As illustrated in FIG. 2, no tension may be desired in such a region as the second wound 226b does not extend to that region of tissue site 220. Additionally, or alternatively, release mechanisms can be used to control tension. For example, release mechanism may include a switch or lock that keeps ratchet mechanism (e.g., pawl 134) in a disengaged state. Accordingly, tension can be controlled and selectively applied to tissue site 220. By controlling tension, wound closure device 210 can be adjusted and may reduce an amount of wound closure devices 210 used for wound closure as compared to non-adjustable wound closure devices.

Referring to FIG. 3, a perspective view of an example of a wound closure device 310 is shown. Wound closure device 310 includes base members 312, 314, and 316 and attachment members 322a-c. Base members 312, 314, 316 may include or correspond to base members 112, 114, 212, 214a, and 214b. Attachment members 322a-c may include or correspond to attachment members 122a-c and 222a-c.

In some implementations, one or more attachment members of attachment members 322a-c include a strain gauge 352 configured to detect and output a strain value. For example, strain generated by tension in a particular attachment member causes a resistance changes (e.g., induces a resistance or change in resistance) that is indicative of a value of the strain, i.e., a strain value. Processing the detected resistance value or the resistance value output by the strain gauge 352 enables determination of the strain value. Strain gauge 352 may include or correspond a semiconductor strain gauge or a foil strain gauge.

Additionally, or alternatively, wound closure device 310 includes or a bandage 332 or is configured to be coupled to a bandage 332. Bandage 332 is configured to protect a wound or opening, such as wound 326a. Bandage 332 may be coupled to one or more base members 312-316, one or more attachment members 322a-c, or a combination thereof. As illustrated in FIG. 3, bandage 332 is coupled to attachment members 322a-c. To illustrated, bandage 332 is woven through attachment members 322a-c such that bandage 332 is between second attachment member 322b and wound 326. In other implementations, bandage 332 is coupled to wound closure device 110 by adhesive, such as LSA 196, or bandage 332 includes or defines openings which are configured to receive one or more attachment members 322a-c prior to insertion into a base member, such as second base member 314.

As illustrated in FIG. 3, tissue site 320 includes a first wound 326a and a second wound 326b. First wound 326a and second wound 326b may include or correspond to surgical openings. The surgical openings may be closed with stiches or a tissue safe topical adhesive, such as Dermabond, a registered trademark of Ethicon. Wounds 326a and/or 326b may include or correspond to wound 126, 226a, or 226b. Tissue site 320 may include or correspond to tissue site 120 or 220.

First base member 312 is positioned proximate to first wound 326a, second base member 314 is positioned between wounds 326a and 326b, and third base member 316 is positioned proximate to second wound 326b. Each of base members are oriented along a first direction 392 and are aligned in a second direction 394. In other implementations, additional base members may be oriented along or with respect to another direction, such as second direction 394. Such a configuration may provide a closing force on the wounds 326a and 326b from multiple directions. Additionally, or alternatively, additional base members may oriented along the first direction and aligned in the second direction. Such a configuration can enable multiple sets of base members to be aligned to treat longer and/or larger wounds.

Attachment members 322a-c couple each of the base members 312, 314, and 316 to each other. To illustrate, each of attachment members 322a and c are attached to first base member 312 and extend through corresponding openings defined by second and third base members 314 and 316. Second attachment member 322b is attached to third base member 316 and extends through corresponding openings defined by first and second base members 312 and 314. As illustrated in FIG. 3, second attachment member 322b is coupled between layers of third base member 316, as described further with reference to FIG. 4B. Such a configuration may enable easier removal of a protective film or light blocking layer from third base member 316 as compared to base members 312 and 314.

Operation of wound closure device 310 is similar to operation of wound closure devices 110 and 210 as described above. When tightening the wound closure device, e.g., inserting or feeding the attachment members 322a-c into openings defined by the base members 312-316, tension is created. A patient or care provider may employ device 318 to determine and/or output a strain measured by strain gauge 352. As illustrated in FIG. 3, strain gauge 352 determines a strain or tension associated with second wound 326b. In some implementations, device 318 reads resistance in the strain gauge, generated by strain, and outputs a measured strain value. The patient or care provide can adjust the tension based on the measured strain value. For example, the patient can tighten the wound closure device 310 by feeding more of an attachment member through an opening, i.e., shortening a length of an attachment member between two base members. As another example, the patient or care provide can loosen the wound closure device 310 by removing attachment member or increasing a length of an attachment member between two base members.

In other implementations, device 318 captures images or video before, during, and/or after tightening to determine deformation, and the patient or care provider adjust tension accordingly. For example, changes in dimension of the strain gauge 352 (or a strain indicator 152) can be captured and determined by device 318. Device 318 can calculate a strain value and/or tension value based on the change in dimensions and output a result indicating the strain value and/or tension value. Thus, wound closure device 310 can include bandages 332 to protect wounds and promote healing and can incorporate strain gauges 352 which may provide a higher degree of accuracy as compared to strain indicators 152.

Wound closure devices 110, 210, and 310 may be combined with dressings (e.g., wound dressings such as dressing 616) to manage fluids (e.g., exudate) in tissue site. For example, a wound dressing may be place over a wound closure device 110, 210, or 310, as described with reference to FIG. 6.

Although FIGS. 1-3 illustrate linear configurations, other configurations of base members and attachment members may be used. For example, a base member may have a shape that matches or mirrors a shape of the wound. To illustrate, curved base members may be used for wounds with curves. As another example, base members and/or attachment members can be arranged radially to provide a tension force to converges to a central area or point. As yet another example, a zig zag or crossing patterns can be used. For example, a single attachment member can attach multiple times to two or more base members to “snake” back and forth across a wound. As an example of crossing patterns, two attachment members may cross one another to provide tension across a wound. Additionally, although FIGS. 1-3 illustrate attachment members as linear racks in other implementations, attachment members include or correspond to screws, rods, etc.

Referring to FIGS. 4A-4F, examples of cross-sections of a base member 402 are shown. For example, base member 402 may include or correspond to one of base members 112, 114, 212, 214a, 214b, 312, 314, or 316. Referring to FIGS. 4A and 4B, exemplary positions of an attachment member, such as attachment member 122a, are illustrated. Referring to FIG. 4A, a cross-section 410 of base member 402 is shown. In FIG. 4A, base member 402 includes layers of attachment member 122a, light blocking layer 492, non-light blocking layer 494, LSA 196, and adhesive cover film 498. Layers 492-496 may include or correspond to layers 192-196 of FIG. 1 respectively.

As illustrated in FIG. 4A, the attachment member 122a is coupled, attached, or bonded to a first side (e.g., top side) of light blocking layer 492 of base member 112. In FIG. 4B, a similar cross-section 412 is shown where the attachment member 122a is coupled, attached, or bonded to a second side (e.g., top side) of light blocking layer 492 of base member 112, such as a side associated with non-light blocking layer 494. Accordingly, attachment member 122a is positioned between the light blocking layer 492 and the non-light blocking layer 494. Such a configuration enables easier removal of non-light blocking layer 494 from the light blocking layer 492 and may enable an entirety of light blocking layer 492 to be removed. For example, as compared to FIG. 4A, the configuration of the base member 112 in FIG. 4B enables a user to remove light blocking layer 492 from base member 112 without removing attachment member 122a or without leaving behind portions of light blocking layer 492 that are between attachment member 122a and non-light blocking layer 494.

Additionally, FIG. 4B illustrates a shroud 424 proximate to or coupled to base member 112. Shroud 424 is positioned such that the shroud blocks the LSA 196 from receiving light, such as ambient light. Shroud 424 is formed of or includes a material that is configured to block (e.g., reflect or absorb) ambient light that would otherwise activate LSA 196. Thus, shroud 424 enables the use of ambient light and/or does not require a dedicated light device, transporting light via components of a therapy system, or both. Accordingly, LSA 196 can be activated without a dedicated light device. In some implementations where shroud 424 or a dressing (e.g., 616) is used, light blocking layer 492 may be optional or may be removed after base member 112 is attached to tissue site and prior to activation of LSA 196. As shroud 424 or dressing will block light, LSA 196 may not need an additional light blocking layer (e.g., 492). Thus, manufacturing can be simplified and costs reduced, by removal of a layer, and/or breathability of the tissue site can be increased by removal of layer that usually has a lower degree of breathability as compared to non-light blocking layer 494.

Referring to FIGS. 4C and 4D, exemplary configurations of light blocking layers 492 are illustrated. Sidewalls 444 are illustrated in FIGS. 4C and 4D and a tab 144 is illustrated in FIG. 4D. Referring to FIG. 4C, a cross-section 414 of base member 402 including a light blocking layer 492 that partially encompasses LSA 196 is shown. To illustrate, light blocking layer 492 has sidewalls (e.g., vertically arranged portions) that extend past the non-light blocking layer 494 and to the LSA 196. FIG. 4D illustrates a cross-section 416 of base member 402 including a light blocking layer 492 that partially encompasses LSA 196 and includes a tab 144. Tab 144 may enable easier removal of light blocking layer 492, particularly sidewalls 444 thereof. The sidewalls 444 of FIGS. 4C and 4D block or filter light (e.g., ambient light) from reaching LSA 196 from the sides of base member 402. Accordingly, such configurations may prevent or reduce unwanted activation and LSA 196 and thus LSA 196 may provide a stronger bond for a longer period of time.

Referring to FIGS. 4E and 4F, exemplary configurations of patterns of LSA 196 on a base member 402 are illustrated. Specifically, configurations where LSA 196 is coated or disposed on a portion of non-light blocking layer 494 (e.g., not coated or disposed on an entirety of non-light blocking layer 494). FIG. 4E illustrates a cross-section of 418 of a base member 402 further including adhesive 496 (e.g., pressure sensitive adhesive) and including a pattern of LSA 196 and adhesive 496. In FIG. 4E, adhesive 496 is positioned in between sections or portions of LSA 196. Adhesive 496 may have a higher bond strength or a lower bond strength as compared to a bond strength of LSA 196. Thus, by selecting different adhesives (e.g., with different bond strengths) and/or using different amounts of adhesives, a bond strength of the base member 402 can be tailored to meet design specifications.

FIG. 4E also illustrates exemplary perforations 452-458. One or more perforations 452-458 can be formed in one or more layers of base member 112 to increase breathability, control a peel strength, or a combination thereof. For example, perforations 452 and 454 may increase breathability by improving breathability in light blocking layer 492. As another example, perforation 456 may increase breathability by improving breathability through layers 492 and 494. As yet another example perforation 458 may increase breathability in one or more layers of layers 196 or 492-496, such as from tissue site to ambient air.

Additionally, or alternatively, perforation 454 may reduce a peel strength between light blocking layer 492 and non-light blocking layer 494 and/or control removal of light blocking layer 492 from non-light blocking layer 494. Similar perforations (e.g., intralayer perforations) for layers 494 and 196 may reduce peel strength between layers 494 or 196 and adjacent layers and may control removal of light blocking layer 492 from non-light blocking layer 494 and of LSA 196 from tissue site. Such perforations may be formed in patterns in the base member 402 and perforations of different layers may be offset from each other to enable selective reduction in peel strength between two layers.

Referring to FIG. 4F, a cross-section 420 of a base member 402 including a pattern of LSA 196 applied to recesses of non-light blocking layer 494 is illustrated. The recesses of non-light blocking layer 494 may be defined by different thicknesses in the non-light blocking layer 494 across cross-section 420. The recesses can be made during formation of non-light blocking layer 494 or formed after formation of non-light blocking layer 494, such as by machining or etching. The LSA 196 can be applied to recesses of non-light blocking layer 494 to reduce a bond strength between base member 402 and tissue sites. Although LSA 196 is employed on edges of base member 402, in other implementations, LSA 196 may be employed on an interior of base member 402 to reduce exposure of the LSA 196 from the side. In other implementations, non-light blocking layer 404 does not include recesses and LSA 196 is applied to portions of non-light blocking layer 494 such that spaces or gaps are defined by discrete portions of sections of the LSA 196.

One or more features of FIGS. 4A-4F may be combined with one or more other features of FIGS. 4A-4F. For example, sidewalls 444 may be added to base members 402 of FIGS. 4E and 4F. Although FIGS. 4A-4F illustrate adhesive cover film 498, adhesive cover film 498 is optional and may not be included in some implementations. As described with reference to FIG. 1A, adhesive cover film 498 is removed prior to use, i.e., adhering LSA 196 to a tissue site.

FIGS. 5A-5C illustrate various examples of visual indications provided by the strain indicator 152 of FIG. 1A to a patient or care provider. FIGS. 5A and 5B depict strain indicator 152 before and after deformation, and FIG. 5C illustrates various examples of icons or markings and how they deform.

Referring to FIGS. 5A and 5B, strain indicator 152 is configured to provide two visual indications. By providing two visual indications, strain indicator 152 can indicate at least two tension states. Strain indicator 152 includes one or more indicia, such a first indicia 502. In some implementations, the indicia (e.g., 502, 504) has a first characteristic in a first state and a second characteristic in a second state. Characteristics may include a color, a shape, an opening, an indentation, a ridge, a projection, etc., or a combination thereof. In other implementations, the indicia has a characteristic with a first value a first state and a second value for the characteristic in a second state. For example when the characteristic corresponds to color, the indicia may have a darker shade when in the first state (e.g., undeformed state) and may have a light shade when in the second state (e.g., deformed state).

As illustrated in FIG. 5A, strain indicator 152 includes a rectangular marking (e.g., icon) as the first indicia 502 and provides a first visual indication to indicate a first tension state when first indicia 502 is visible. For example, the first tension state may include or correspond to a non-operational state and indicate a tension of less than or equal to a threshold tension, as described with reference to FIG. 1A.

Referring to FIG. 5B, the strain indicator 152 of FIG. 5A deforms as attachment member 122b is inserted into a base member to couple two base members together and generate tension. Strain indicator 152 deforms under the generated tension and indicia 502 deforms to form second indicia 504. As illustrated in FIG. 5B, indicia 502 is a rectangular marking which deforms to form a square marking, indicia 504. The strain indicator 152 provides a second visual indication to indicate a second tension state when the second indicia 504 (e.g., square marking) is visible. For example, strain indicator 152 may not deform such that the first indicia 502 is visible to indicate a first tension state of relaxed, under-tension, or non-operational tension and may deform under tension such that the second indicia 504 is visible to indicate a second tension state of operational tension, such as a tension of greater than or equal to a threshold tension, as described with reference to FIG. 1A.

Additionally, or alternatively, strain indicator 152 produces a color change upon deforming. In some implementations, the color change is achieved by adding a liquid crystal material or component to the polymer material of the strain indicator 152. The liquid crystal material or component provides a color change upon deformation (i.e., changes color as it deforms) and reducing in thickness. In other implementations, deformation of the strain indicator 152, such as by lengthening or reducing in thickness produces a color change that is visible to a human or measureable by a device (e.g., a camera or sensor). Thus, a visual indication may indicate a tension of a tissue site being greater than or equal to a tension threshold associated with the corresponding tension state.

In some implementations, strain indicator 152 is further configured to provide a third visual indication. For example, strain indicator 152 may indicate a third tension state of excess tension or over tension by producing the third visual indication. To illustrate a second rectangle or another shape may be used to indicate the third tension state. As another example, a color change may be used as the third visual indication to indicate the third state. The color change may be produced by an additive or component of the polymer of the strain indicator, such as a liquid crystal material or component. As yet another example, second indicia 504 may deform further to produce a third indicia. To illustrate, with regards to the indicia 502, 504 of FIGS. 5A and 5B, an exemplary third indicia includes a rectangle icon or marking with an aspect ratio that is opposite indicia 502, i.e., 2:1 rather than 1:2.

Although the strain indicator 152 has been described and/or shown as having a rectangular icon (or marking) that deforms to a square icon under tension in FIGS. 5A and 5B, in other implementations, strain indicator 152 may have an icon other than a rectangle, such as an oval, circle, square, triangle, quadrilateral, pentagon, star, or another shape, as illustrative, non-limiting examples. Referring to FIG. 5C, other examples of icons of strain indicator 152 are illustrated. In FIG. 5C, shapes 512-518 undergo a left to right deformation under tension to produce a corresponding shape 522-528. Thus, as illustrated in FIG. 5C, shapes 512-518 other than a rectangle can be used to produce a visual indication, such as a triangle 512, a square 514, a parallelogram 516, and an oval 518. The triangle 512 may be an isosceles triangle that deforms to an equilateral triangle 522. Alternatively, the square 514 can deform to a rectangle 524 to provide multiple visual indications. For example, when the rectangle 524 is has an a first aspect ratio (e.g., 2:1) the icon indicates an operational state and when the rectangle 524 has a second aspect ratio (e.g., 3:1) the icon indicates an over tensioned state. The parallelogram 516 may deform to a rhombus 526 or a square, and the oval 518 (or ellipse) may deform to a circle 528, as illustrated in FIG. 5C.

In some implementations, a color change or deformation of an icon can be read by an external device, such as a therapy device (e.g., 610) or a mobile phone (e.g., 318). The therapy device or mobile phone may use reference icons and comparisons to determine deformation and strain values or states. For example, a reference icon may be provided on the attachment member and compared with the deformed icon of the strain indicator 152. As another example, the therapy device or mobile phone can determine a deformation amount, size, and/or aspect ratio of a deformed icon by capturing images before and after deformation or during deformation.

Although FIGS. 5A-5C illustrate and described multiple separate configurations, aspects of each configuration can be used separately or in combination with aspects of other configurations. The visual indications and states of FIGS. 5A-5C may have tension ranges and thresholds as described with reference to FIG. 1A. For example, a first visual indication is provided when exposed to a tension (e.g., 4 N) within the first range of tensions and a second visual indication is provided when exposed to a second tension (e.g., 8 N) within the second range of tensions.

In addition to or in the alternative of indicia 502, 504, changes in dimensions of strain indicator 152 can be used to determine a tension state. In such implementations, a person or a device (e.g., 318) can determine a change in length 506, a change in width 508, a change in thickness, or a combination thereof, of the strain indicator 152 during deformation. For example, reducing thickness or increasing length by one-half or one-third may produce a first visual indication and reducing thickness by another one-half or one third may produce a second visual indication. As illustrated in FIGS. 5A and 5B the change in length 506 and the change in width 508 correspond to the strain indicator 152. In other implementations, the change in length 506 and the change in width 508 correspond to the indicia 502, 504 of the strain indicator 152, such as changes in dimensions of the rectangular icon.

FIG. 6 shows a perspective view of an illustrative system 600 (e.g., a therapy system) for providing wound therapy. System 600 may include a wound closure device as described herein (e.g., 110, 210, 310), a therapy device 610, a canister 612, a tube 614, a dressing 616, and a light source 618 (e.g., UV device). System 600 is configured to provide therapy (e.g., oxygen therapy, positive-pressure therapy, negative-pressure therapy, or a combination thereof) at a tissue site 620 associated with a target area of a patient. For example, dressing 616 may be in fluid communication with tissue site 620 and may be in fluid communication with therapy device 610 via tube 614. Light source 618 may include or correspond to light source 118 or UV device 119 of FIG. 1. In some implementations, system 600 may include one or more components commercially available through and/or from KCI USA, Inc. of San Antonio, Tex., U.S.A., and/or its subsidiary and related companies (collectively, “KCI”).

Therapy device 610 (e.g., a treatment apparatus) is configured to provide therapy to tissue site 620 via tube 614 and dressing 616. For example, therapy device 610 may include a pressure source (e.g., a negative-pressure source, such as a pump, or a positive-pressure source, such as a pressurized oxygen container, an oxygen concentrator, or an oxygen collector) configured to be actuatable (and/or actuated) to apply pressure differential relative to ambient conditions to dressing 616. As illustrative, non-limiting examples, positive-pressure applied to a tissue site may typically ranges between 5 millimeters mercury (mm Hg) (667 pascals (Pa)) and 30 mm Hg (4.00 kilo (k) Pa). Common therapeutic ranges are between 10 mm Hg (1.33 kPa) and 25 mm Hg (3.33 kPa). As illustrative, non-limiting examples, reduced-pressure applied to a tissue site may typically ranges between −5 millimeters mercury (mm Hg) (−667 pascals (Pa)) and −500 mm Hg (−66.7 kilo (k) Pa). Common therapeutic ranges are between −75 mm Hg (−9.9 kPa) and −300 mm Hg (−39.9 kPa).

In some implementations, therapy device 610 may alternate between providing positive-pressure therapy and negative-pressure therapy to the dressing 616, may provide positive-pressure therapy to a first portion of the dressing 616 and negative-pressure therapy to a second portion of the dressing 616, may provide no positive or negative pressure, or a combination thereof. In some such implementations, the therapy device 610 can provide positive-pressure therapy and negative-pressure therapy to the dressing 616 at the same time (e.g., partially concurrently).

As illustrated in FIG. 6, therapy device 610 includes canister 612 to receive fluid from tissue site 620 or to provide fluid to tissue site 620. Although canister 612 is illustrated as being internal to and/or integrated with therapy device 610, in other implementations, canister 612 is external to therapy device 610, as illustrated and described with reference to FIG. 1A.

Therapy device 610 may also include one or more other components, such as a sensor, a processing unit (e.g., a processor), an alarm indicator, a memory, a database, software, a display device, a user interface, a regulator, and/or another component, that further facilitate positive-pressure therapy. Additionally, or alternatively, therapy device 610 may be configured to receive fluid, exudate, and or the like via dressing 616 and tube 614. Therapy device 610 may include one or connectors, such as a representative connector 638. Connector 630 is configured to be coupled to tube 614. Additionally, or alternatively, therapy device 610 may include one or more sensors, such a pressure sensor (e.g., a pressure transducer). The one or more sensors may be configured to enable therapy device 610 to monitor and/or sense a pressure associated with tube 614 and/or dressing 616.

Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

In some implementations, tube 614 may include multiple lumens, such as a primary lumen (e.g., a positive-pressure/fluid lumen) for application of positive-pressure and/or communication of fluid, and one or more secondary lumens proximate to or around the primary lumen. The one or more secondary lumens (e.g., one or more ancillary/peripheral lumens) may be coupled to one or more sensors (of therapy device 610), coupled to one or more valves, as an illustrative, non-limiting example. Although tube 614 is described as a single tube, in other implementations, system 600 may include multiple tubes, such as multiple distinct tubes coupled to therapy device 610, dressing 616, or both.

As used herein, a “tube” broadly refers to a tube, pipe, hose, conduit, or other structure with one or more lumens adapted to convey fluid, exudate, and/or the like, between two ends. In some implementations, a tube may be an elongated, cylindrical structure with some flexibility; however, a tube is not limited to such a structure. Accordingly, tube may be understood to include a multiple geometries and rigidity. Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

Dressing 616 includes a connector 630 (also referred to as a dressing connection pad or a pad), a drape 632, and a manifold 634 (also referred to as a distribution manifold or an insert). Drape 632 may be coupled to connector 630. To illustrate, drape 632 may be coupled to connector 630 via an adhesive, a separate adhesive drape over at least a portion of connector 630 and at least a portion of drape 632, or a combination thereof, as illustrative, non-limiting examples.

Drape 632 may be configured to couple dressing 616 at tissue site 620 and/or to provide a seal to create an enclosed space (e.g., an interior volume) corresponding to tissue site 620. For example, drape 632 may be configured to provide a fluid seal between two components and/or two environments, such as between a sealed therapeutic environment and a local ambient environment. To illustrate, when coupled to tissue site 620, drape 632 is configured to maintain a pressure differential (provided by a positive-pressure source or a negative-pressure source) at tissue site 620. Drape 632 may include a drape aperture that extends through drape 632 to enable fluid communication between device and target tissue. Drape 632 may be configured to be coupled to tissue site 620 via an adhesive, such as a medically acceptable, pressure-sensitive adhesive that extends about a periphery, a portion, or an entirety of drape 632. Additionally, or alternatively, drape 632 may be coupled to tissue site 620 via a double-sided drape tape, paste, hydrocolloid, hydrogel, and/or other sealing device or element, as illustrative, non-limiting examples.

Drape 632 may include an impermeable or semi-permeable, elastomeric material, as an illustrative, non-limiting example. In some implementations, drape 632 may be liquid/gas (e.g., moisture/vapor) impermeable or semi-permeable. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In some implementations, drape 632 may include the “V.A.C.® Drape” commercially available from KCI. Additional, specific non-limiting examples of materials of drape 632 may include a silicone drape, 3M Tegaderm® drape, and a polyurethane (PU) drape such as one available from Avery Dennison Corporation of Pasadena, Calif. An additional, specific non-limiting example of a material of the drape 632 may include a 30 micrometers (μm) matt polyurethane film such as the Inspire™ 2317 manufactured by Exopack™ Advanced Coatings of Matthews, N.C.

Manifold 634 is configured to be positioned on and/or near tissue site 620, and may be secured at the tissue site 620, such as secured by drape 632. The term “manifold” as used herein generally refers to a substance or structure that may be provided to assist in applying a pressure differential (e.g., positive-pressure differential) to, delivering fluids to, or removing fluids and/or exudate from a tissue site and/or target tissue. The manifold typically includes a plurality of flow channels or pathways that distribute fluids provided to and removed from the tissue site. In an illustrative implementation, the flow channels or pathways are interconnected to improve distribution of fluids provided to or removed from the tissue site. Manifold 634 may be a biocompatible material that may be capable of being placed in contact with the tissue site and distributing positive and/or negative-pressure to the tissue site. Manifold 634 may include, without limitation, devices that have structural elements arranged to form flow channels, such as foam, cellular foam, open-cell foam, porous tissue collections, liquids, gels, and/or a foam that includes, or cures to include, flow channels, as illustrative, non-limiting examples. Additionally, or alternatively, manifold may include polyethylene, a polyolefin, a polyether, polyurethane, a co-polyester, a copolymer thereof, a combination thereof, or a blend thereof.

In some implementations, manifold 634 is porous and may be made from foam, gauze, felted mat, or other material suited to a particular biological application. In a particular implementation, manifold 634 may be a porous foam and may include a plurality of interconnected cells or pores that act as flow channels. The foam (e.g., foam material) may be either hydrophobic or hydrophilic. As an illustrative, non-limiting example, the porous foam may be a polyurethane, open-cell, reticulated foam such as GranuFoam® material manufactured by Kinetic Concepts, Incorporated of San Antonio, Tex.

In some implementations, manifold 634 is also used to distribute fluids such as medications, antibacterials, growth factors, and other solutions to the tissue site. Other layers may be included in or on manifold 634, such as absorptive materials, wicking materials, hydrophobic materials, and hydrophilic materials. In an implementation in which the manifold 634 includes a hydrophilic material, manifold 634 may be configured to wick fluid away from tissue site 620 and to distribute positive-pressure to tissue site 620. The wicking properties of manifold 634 may draw fluid away from the tissue site 620 by capillary flow or other wicking mechanisms. An illustrative, non-limiting example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether and/or foams that have been treated or coated to provide hydrophilicity.

In some implementations, manifold 634 is constructed from bioresorbable materials that do not have to be removed from tissue site 620 following use of the system 600. Suitable bioresorbable materials may include, without limitation, a polymeric blend of polylactic acid (PLA) and polyglycolic acid (PGA). The polymeric blend may also include without limitation polycarbonates, polyfumarates, and capralactones. Manifold 634 may further serve as a scaffold for new cell-growth, or a scaffold material may be used in conjunction with manifold 634 to promote cell-growth. A scaffold may be a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Although a manifold 634 is illustrated in FIG. 6, in other implementations, dressing 616 does not include manifold 634. In such implementations, drape 632 of dressing 616 is coupled to connector 630.

Connector 630 includes a body 642 (e.g., housing) and a base 644, and is configured to be coupled to tube 614 via an interface 646 (e.g., a port). Base 644 is configured to be coupled to dressing 616. For example, base 644 may be coupled, such as via an adhesive, to drape 632 and/or manifold 634. In some implementations, base 644 comprises a flange that is coupled to an end of body 642 and/or is integrally formed with body 642. Connector 630, such as body 642, base 644, interface 646, or a combination thereof, may be made of rigid material and/or a semi-rigid material. In a non-limiting example, connector 630 may be made from a plasticized polyvinyl chloride (PVC), polyurethane, cyclic olefin copolymer elastomer, thermoplastic elastomer, poly acrylic, silicone polymer, or polyether block amide copolymer. In some implementations, connector 630 is formed of a semi-rigid material that is configured to expand when under a force, such as positive-pressure greater than or equal to a particular amount of pressure. Additionally or alternatively, connector 630 may be formed of a semi-rigid material that is configured to collapse when under a force, such as reduced-pressure less than or equal to a threshold pressure.

Body 642 includes one or more channels or one or more conduits that extend from and/or are coupled to interface 646. To illustrate, body 642 may include a primary channel configured to be coupled in fluid communication with a primary lumen (e.g., 621) of tube 614. The primary channel may be coupled to a cavity (e.g., a tissue cavity partially defined by body 642) having an aperture open towards manifold 634 (and/or towards tissue site 620). For example, the primary channel may include a first opening associated with interface 646 and a second opening (distinct from the aperture of the cavity) associated with the cavity. Thus, the primary channel may define a through channel of body 642 to enable fluid communication between interface 646 and tissue site 620.

Body 642 includes a channel (e.g., a through channel) having a first aperture open opposite dressing 616 and a second aperture open towards dressing 616. For example, the first aperture is located on an outer surface side (e.g., an ambient environment surface) of connector 630 and the second aperture is located on an inner surface side (e.g., a tissue facing side) of connector 630. The second aperture is configured to be coupled to one or more lumens of tube 614, such as coupled via the cavity. Illustrative, non-limiting examples of commercially available connectors include a “V.A.C. T.R.A.C.® Pad,” or “Sensa T.R.A.C.® Pad” available from Kinetic Concepts, Inc. (KCI) of San Antonio, Tex.

In some implementations. dressing 616 further includes viewing member 650. As illustrated in FIG. 6, viewing member 650 is coupled to or integrated with body 642 or comprises a portion of body 642 of connector 630. Viewing member 650 is configured to enable viewing of wound or opening 126, strain indicator 152 or both. For example, viewing member 650 is positioned in a channel defined in or by connector or on top of the channel. In some implementations, viewing member 650 includes or corresponds to a transparent material or a translucent material such that wound or opening (e.g., 126), strain indicator 152, or both, are visible through viewing member 650. In a particular implementation, viewing member 650 includes a cover or flap to protect tissue site 620 and wound closure device 110 from ambient light.

During operation of system 600, wound closure device 110 is coupled to tissue site 620 proximate to a wound (e.g., 126) as described with reference to FIG. 1A. Dressing 616 is coupled to tissue site 620 over wound closure device 110. Additionally, dressing 616 is coupled to device 610 via tube 614. A pressure differential, such as positive-pressure, can be generated and/or applied to dressing 616 (e.g., an interior volume of dressing 616) by a pressure source associated with device 610. When positive-pressure is generated and/or applied to dressing 616, fluid or medication from device 610, such as from canister 612, may be transported to dressing 616. Furthermore, in some implementations, reduced-pressure can be applied to dressing 616 (e.g., the interior volume of dressing 616 or a second interior volume of the dressing 616) by a reduced-pressure source associated with device 610. When reduced-pressure is applied to dressing 616 (e.g., when vacuum pressure is generated, fluid, exudate, or other material within dressing 616 may be transported to canister 612 of device 610.

During operation of therapy, patient or care provide can inspect tissue site 620 (e.g., wound thereof) and/or wound closure device 110 (e.g., strain indicator 152 thereof) to determine if an adjustment (e.g., increase or decrease tension) to wound closure device 110 should be made. If a determination indicates an adjustment is to be made, dressing 616 can be removed to access and adjust wound closure device 110. Dressing 616 may be reapplied or another dressing may be applied to tissue site 620 and therapy may resume.

After operation, such as completion of therapy, system 600 may be disconnected and removed from tissue site 620. Wound closure device 110 may be removed by activating a light source and exposing LSA thereof to light, as described with reference to FIG. 1A.

Thus, wound closure device 110 can be incorporated into a therapy system and operate in conjunction with a wound dressing. Accordingly, therapy and wound closing tension can be applied to a wound to increase healing times and reduce infections.

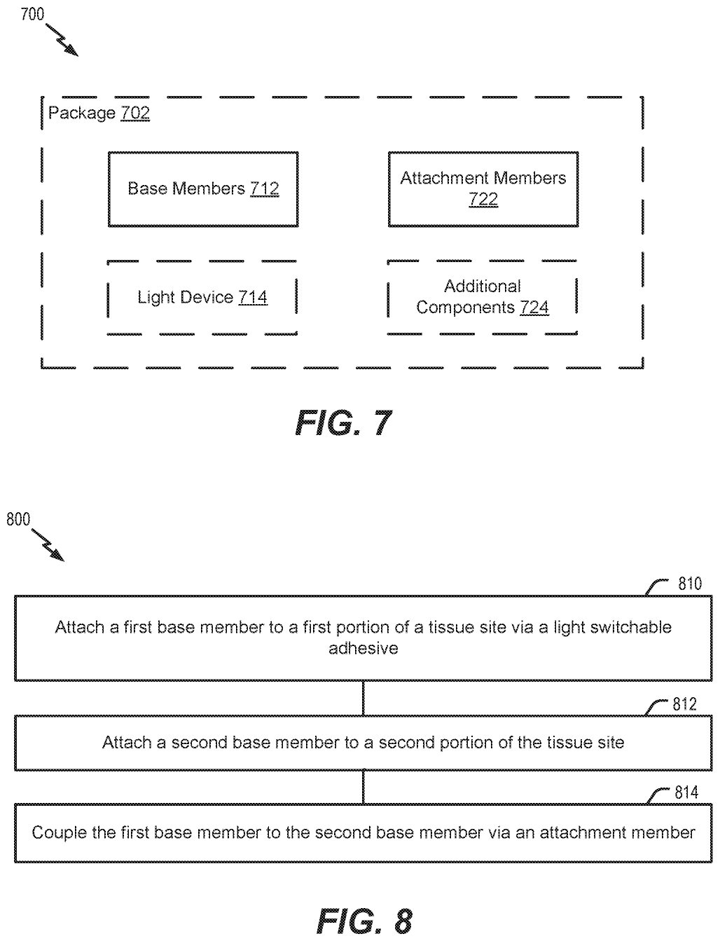

Referring to FIG. 7, a kit 700 for wound closure devices, such as wound closure device 110 of FIG. 1A, is illustrated. Kit 700 includes one or more base members 712 and/or one or more attachment members 722. The one or more base members 712 may include or correspond to base members 112, 114 of FIG. 1A, base members 212, 214a, 214b of FIG. 2, base members 312, 314, 316 of FIG. 3, base member 402 of FIG. 5, one or more other base members, or a combination thereof. The one or more attachment members 722 may include or correspond to attachment member(s) 122a-c of FIG. 1A, attachment member(s) 222a-c of FIG. 2, attachment member(s) 322a-c of FIG. 3, one or more other attachment members, or a combination thereof.

In some implementations, the one or more attachment members 722 include a strain gauge or a strain indicator, as described herein. In a particular implementation, the one or more attachment members 722 include teeth going in multiple directions. For example, the one or more attachment members 722 include teeth (e.g., 132) in two directions. The one or more attachment members 722 can be inserted into openings (e.g., 142) of a first base member (e.g., 214a) and a second base member (e.g., 214b) from the inside or proximate to the wound, and fed through corresponding openings of both base members outwards and away from the wound. To illustrate, the one or more attachment members 722 include two linear racks that are opposite each other to facilitate engaging with a respective pawl (e.g., 134) in each base member. Thus, the one or more attachment members 722 may not be anchored or coupled to a base member and may be inserted into openings of two base members to couple a particular attachment member to multiple base members, as described with reference to FIG. 1.

Additionally, the one or more attachment members 722, such as attachment members including strain indicators and/or strain gauges, can be used with other means of attachment and/or independent of base members. For example, the one or more attachment members 722 can be coupled to conventional adhesive strips or tissue hooks and can indicate a tension applied to a wound by the adhesive strips/tissue hooks and the one or more attachment members 722.

In some implementations, kit 700 may also include a light device 714, one or more additional components 724, or a combination thereof. The light device 714 may include or correspond to a light source 118, a UV device 119, or a combination thereof. The one or more additional components 724 may include or correspond to LSA (e.g., 196, such as a tube LSA), an LSA applicator, gloves, antiseptic, medical adhesive, and/or other components. Additionally, or alternatively, the one or more additional components 724 may include strain gauges (e.g., 152), strain indicators (e.g., 352), devices (e.g., 314), or a combination thereof.

In some implementations, kit 700 may include a package 702. For example, package 702 may include a box, a bag, a container, or the like. Package 702 may include the one or more base members 712 and the one or more attachment members 722. In some implementations, package 702 may include the light device 714, the one or more additional components 724, or a combination thereof. Additionally, or alternatively, package 702 may include a packaging medium (e.g., packaging material), such as foam, paper, or the like

In a particular implementation, kit 700 for a wound closure device is disclosed, such that the wound closure device, when adhered to target tissue of a tissue site proximate to a wound, is configured to support closing of the wound and promote healing of the wound. In some implementations, kit 700 includes two or more different types of base members 712 that, when coupled together by an attachment member, generate tension to be close a wound.

Thus, FIG. 7 describes kit 700 for an apparatus (e.g., a wound closure device) that includes a plurality of base members which may be coupled together by attachment member(s) to secure the apparatus to a tissue site by LSA. Additionally, or alternatively, the one or more attachment members may provide a guide for generating tension that would otherwise be unavailable in conventional wound closure devices. For example, as an attachment member is fed through a base member, a strain indicator of the attachment member may advantageously provide a visual indication of tension to guide setup of and/or adjustments to the wound closure device.

FIG. 8 illustrates a method 800 of attaching and using a wound closure device. The method 800 may be performed at, by, or with a wound closure device, such as wound closure device 110, 210, 310. In some implementations, the wound closure device may be included in a wound closure system, such as system 100, or a therapy system (e.g., system 600).

Method 800 includes attaching a first base member to a first portion of a tissue site via a light switchable adhesive, at 810. The first base member may include or correspond to base member 112, 114, 212, 214a, 214b, 312, 314, 216, or 402. First portion of tissue site may include or correspond to target tissue of tissue site 120, 220, 320, or 620, and light switchable adhesive may include or correspond to LSA 196. For example, cover film 198 may be removed from base member 112 and base member 112 may be pressed into target tissue near wound or opening 126.

Method 800 also includes, attaching a second base member to a second portion of the tissue site, at 812. For example, the second base member may include or correspond to another of base member 112, 114, 212, 214a, 214b, 312, 314, 216, or 402. To illustrate, base member 114 may be pressed into target tissue near wound or opening 126 on an opposite side relative to first base member.

In a particular implementation, the base members 112 and 114 are included in or correspond to one sheet of base members or a strip or roll of base member material that can be cut or ripped into a desired size. For example, a patient or care provider can cut a desired length of material for base member 112, base member 114, or both, and then attach the base members 112, 114 to the patient.

Method 800 further includes, coupling the first base member to the second base member via an attachment member, at 814. For example, the attachment member may include or correspond to attachment member 122a-c, 222a-c, or 322a-c. To illustrate, after first and second base member are attached, attachment member 122a is inserted into a corresponding opening 142 defined by or in second base member 114.

Thus, method 800 describes operation of attaching a wound closure device including LSA to a patient. Therefore, by inclusion of LSA and its low tact/peel strength state, a patient or care provider can easily remove the wound closure device (e.g., base members thereof) from the tissue site without damaging the skin and with reduced pain as compared to high peel strength adhesives or hooks. Thus, method 800 describes attaching a wound closure device, and the wound closure device may be configured to ensure effective, efficient, and safe wound closure therapy.

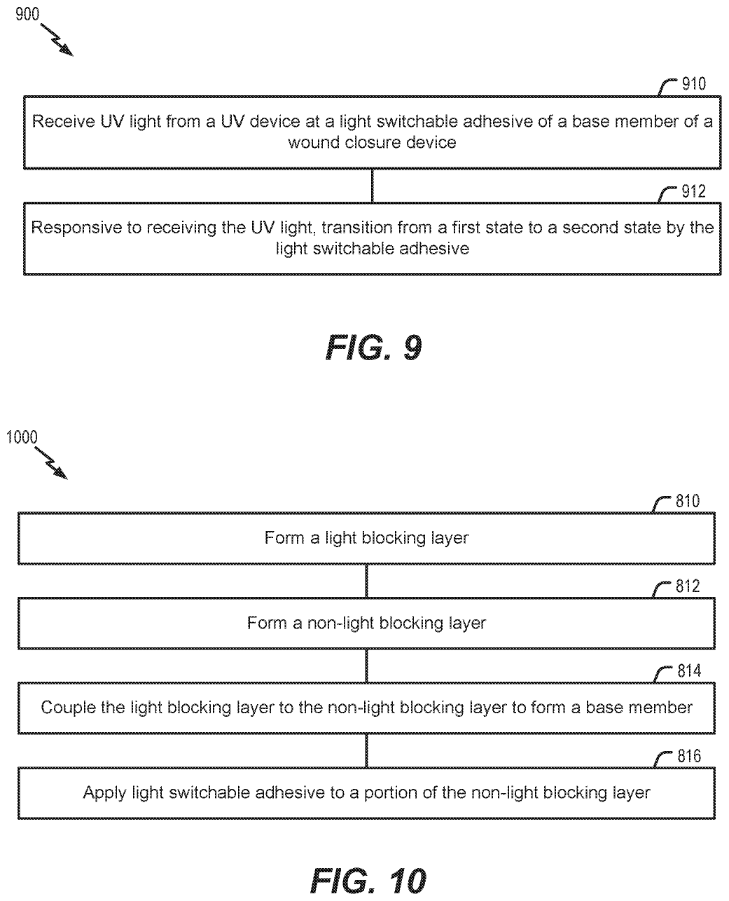

FIG. 9 illustrates a method 900 of operating a wound closure device. The method 900 may be performed at or by system 100 (e.g., wound closure device 110), a system that includes a wound closure device (e.g., 110, 210, 310), or the system 600 (e.g., wound closure device 110).

Method 900 includes receiving UV light from a UV device at a light switchable adhesive of a base member of a wound closure device, at 910. For example, the UV light and UV device may include or correspond to UV light from light source 118, UV device 119, or light source 618. The light switchable adhesive may include or correspond LSA 196. The base member may include or correspond base member 112, 114, 212, 214a, 214b, 312, 314, 216, or 402. To illustrate, protective film 192, shroud 424, light blocking layer 492, or a combination thereof is removed from base member and/or tissue site such that light from the light source is received at LSA 196. In some implementations, the light is received at a first layer (e.g., polymer layer 194 or non-light blocking layer 494) and the light passes through to LSA 196 or the light is received at the first layer and is diffused by the first layer to LSA 196. Alternatively, a port or window, such as 650, may be incorporated into a dressing (e.g., 616) associated with the wound closure device or into a component (e.g., base member) of the wound closure device. The port receives the UV device or light from the UV device passes through the window and is received at LSA 196.

Method 900 further includes, responsive to receiving the UV light, transitioning from a first state to a second state by the light switchable adhesive, at 912. For example, the first state may include or correspond to a tacky state or a high tack state, and the second state may include or correspond to a low tacky state or a non-tacky state. The first state has a high peel strength relative to a peel strength of the second state. To illustrate, photo initiators of LSA 196 absorb the UV light and cross-link, causing the LSA 196 to become brittle and lose peel strength. Therefore, a patient or care provider can easily remove the wound closure device (e.g., base members thereof) from the tissue site without damaging the skin and with reduced pain as compared to high peel strength adhesives or hooks. Thus, method 900 describes operation of a wound closure device, and the wound closure device may be configured to ensure effective, efficient, and safe wound closure therapy.

FIG. 10 illustrates a method 1000 of manufacturing a wound closure device. The method 1000 may be performed to form a wound closure device, such as wound closure device 110, 210, 310. In some implementations, the wound closure device may be included in a wound closure system, such as system 100, or a therapy system (e.g., system 600).

Method 1000 includes forming a light blocking layer, at 1010. For example, the light blocking layer may include or correspond to an attachment member (e.g., 122a), protective film 192, cover film 198, light blocking layer 492, or adhesive cover film 498.

Method 1000 also includes, forming a non-light blocking layer, at 1012. For example, the non-light blocking layer may include or correspond to an attachment member (e.g., 122a), polymer layer 194, or non-light blocking layer 494.

Method 1000 includes, coupling the light blocking layer to the non-light blocking layer to form a base member, at 1014. For example, base member may include or correspond base member 112, 114, 212, 214a, 214b, 312, 314, 216, or 402. To illustrate, the light blocking layer and the non-light blocking layer are co-extruded together.

Method 1000 further includes, applying light switchable adhesive to a portion of the non-light blocking layer, at 1016. For example, the light switchable adhesive may include or correspond to LSA 196, and the light switchable adhesive may be applied to the non-light blocking layer as described with reference to FIGS. 4A-4F.

Thus, method 1000 describes a method of forming a wound closure device including light switchable adhesive. The light switchable adhesive can be activated to transition to a low tact and/or peel strength state to enable easier removal as compared to a peel strength of the light switchable adhesive when in use. Therefore, a patient or care provider can easily remove the wound closure device (e.g., base members thereof) from the tissue site without damaging the skin and with reduced pain as compared to high peel strength adhesives or hooks. Thus, method 900 describes operation of a wound closure device, and the wound closure device may be configured to ensure effective, efficient, and safe wound closure therapy.

It is noted that one or more operations described with reference to one of the methods of FIGS. 8-10 may be combined with one or more operations of another of FIGS. 8-10. For example, one or more operations of method 800 may be combined with one or more operations of method 900. Additionally, or alternatively, one or more operations described above with reference to FIGS. 1A, 1B, 2, 3, 4A-4F, 5A-5C, and 6 may be combined with one or more operations of FIG. 8, FIG. 9, FIG. 10, or a combination of FIGS. 8-10.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively

1. A wound closure device comprising:
a plurality of base members configured to adhere to a tissue site, the plurality of base members include a first base member including light switchable adhesive configured to transition from a first state to a second state, and the light switchable adhesive has a first peel strength in the first state that is greater than a second peel strength of the light switchable in the second state; and
one or more attachment members configured to couple to two or more base members of the plurality of base members.

2. The wound closure device of claim 1, wherein the first base member further includes a pawl, and wherein a particular attachment member of the one or more attachment members comprises a plurality of teeth configured to interlock with the pawl.

3. The wound closure device of claim 2, wherein the particular attachment member is coupled to a second base member of the plurality of base members.

4. The wound closure device of claim 3, wherein the particular attachment member is removably coupled to a second base member of the plurality of base members.

5. The wound closure device of claim 1, the first base member further comprising:
a polymer layer coupled to the light switchable adhesive and configured to pass light to the light switchable adhesive to transition the light switchable adhesive from the first state to the second state; and
a protective film removably coupled to the polymer layer and configured to block ambient light from the light switchable adhesive.

6. The wound closure device of claim 5, wherein the polymer layer is optically transparent.

7. The wound closure device of claim 5, wherein the polymer layer is configured to diffuse the light to pass the light to the light switchable adhesive.

8. The wound closure device of claim 5, wherein the polymer layer is configured to pass light having a wavelength between 10 nanometers and 500 nanometers.

9. The wound closure device of claim 5, wherein the protective film is configured to block or filter light having a wavelength between 10 nanometers and 500 nanometers.

10. The wound closure device of claim 1, wherein the one or more attachment members comprise acrylonitrile butadiene styrene (ABS).

11. The wound closure device of claim 1, further comprising an indicator configured to provide a first visual indication when tension associated with a particular attachment member of the one or more attachment members is greater than or equal to a first threshold.

12. The wound closure device of claim 11, wherein the particular attachment member includes the indicator, wherein the indicator comprises an elastic polymer, and wherein the indicator is configured to deform responsive to a force resulting from feeding the particular attachment member into at least one base member of the plurality of base members.

13. The wound closure device of claim 12, wherein:
the indicator is configured to deform to transition between a first state and a second state,
when the indicator is in the first state, the indicator is configured to indicate a first tension state, and
when the indicator is in the second state, the indicator is configured to indicate a second tension state.

14. The wound closure device of claim 13, wherein the first state comprises an undeformed state, and wherein the second state comprises a deformed state.

15. The wound closure device of claim 13, wherein the indicator has a first color or marking in the first state and a second color or marking different from the first color or marking in the second state.

16. The wound closure device of claim 13 wherein the indicator is configured to provide a second visual indication when the tension of the particular attachment member is greater than or equal to a second threshold.

17. The wound closure device of claim 13, wherein:
the indicator is configured to deform to transition between the second state and a third state,
the indicator has a third color or marking that is different from the first color or marking and the second color or marking, and
the first state corresponds to a first range of tensions, the second state corresponds to a second range of tensions, and the third state corresponds to a third range of tensions.

18. The wound closure device of claim 13, wherein the second visual indication indicates an over-tension state.

19. The wound closure device of claim 11, wherein the indicator is configured to transition to the second state between 4-8 Newtons.

20. The wound closure device of claim 11, wherein the indicator includes a material that has a hardness in the range of 50 Shore A to 80 Shore A.

21. The wound closure device of claim 11, further comprising a second indicator configured to deform to indicate a third tension state.

22. The wound closure device of claim 21, wherein the indicator and the second indicator are integrated into the particular attachment member, and wherein the indicator comprises a first portion of the particular attachment member and the second indicator comprises a second portion of the particular attachment member.

23. The wound closure device of claim 1, wherein a particular attachment member of the one or more attachment members comprises a strain gauge configured to output a strain value of the particular attachment member.

24. The wound closure device of claim 23, further comprising a layer removably coupled to a particular side of the light switchable adhesive, the particular side of the light switchable adhesive configured to couple to the tissue site.

25. The wound closure device of claim 24, further comprising a shroud configured to be coupled to a patient and configured to block ambient light from the light switchable adhesive.

26. The wound closure device claim 25, wherein the first base member further includes adhesive.

27. The wound closure device of claim 26, the plurality of base members including a second base member that includes second light switchable adhesive.

28-45. (canceled)

46. A method of forming a wound closure device, the method comprising:
forming a light blocking layer;
forming a non-light blocking layer;
coupling the light blocking layer to the non-light blocking layer to form a base member; and
applying light switchable adhesive to a portion of the non-light blocking layer.

47. The method of claim 46, further comprising coupling an attachment member to the base member.

48. The method of claim 47, wherein coupling the attachment member to the base member includes bonding to the base member by an adhesive.

49. The method of claim 47, wherein coupling the attachment member to the base member includes bonding the attachment member to the base member by a molding process or forming the attachment member on the base member.

50. The method of claim 47, wherein coupling the attachment member to the base member includes coupling the attachment member to the light blocking layer.

51. The method of claim 47, wherein coupling the attachment member to the base member includes coupling the attachment member between the light blocking layer and the non-light blocking layer.

52-56. (canceled)

57. A kit for an wound closure device, the kit comprising:
one or more attachment members configured to couple to two or more base members, the one or more attachment members including an indicator configured to provide a first visual indication when tension associated with the particular attachment member is greater than or equal to a first threshold.

58. The kit of claim 57, further comprising a package that includes the one or more attachment members and a plurality of base members configured to adhere to a tissue site, the plurality of base members include a first base member including light switchable adhesive, the light switchable adhesive configured to transition from a first state to a second state.

59-64. (canceled)
